SEQUENCE LISTING (41 sequences)

SEQ ID NO: 1 - protein, 1273 aa, artificial sequence
  Sars CoV 2 spike protein of fragment and/or mutant and/or concatemer
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1198 aa, artificial sequence
  Sars CoV 2 spike protein of fragment and/or mutant and/or concatemer
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 3 - protein, 273 aa, artificial sequence
  Sars CoV 2 spike protein of fragment and/or mutant and/or concatemer
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 4 - protein, 201 aa, artificial sequence
  Sars CoV 2 spike protein of fragment and/or mutant and/or concatemer
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 5 - protein, 127 aa, artificial sequence
  Sars CoV 2 spike protein of fragment and/or mutant and/or concatemer
YECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVK

SEQ ID NO: 6 - protein, 17 aa, artificial sequence
  Sars CoV 2 spike protein of fragment and/or mutant and/or concatemer
LPDPSKPSKRSFIEDLL

SEQ ID NO: 7 - protein, 18 aa, artificial sequence
  Sars CoV 2 spike protein of fragment and/or mutant and/or concatemer
QTNSPRRARSVASQSIIA

SEQ ID NO: 8 - protein, 11 aa, artificial sequence
  Sars CoV 2 spike protein of fragment and/or mutant and/or concatemer
SNNLDSKVGGN

SEQ ID NO: 9 - protein, 10 aa, artificial sequence
  Sars CoV 2 spike protein of fragment and/or mutant and/or concatemer
QPTNGVGYQP

SEQ ID NO: 10 - protein, 11 aa, artificial sequence
  Sars CoV 2 spike protein of fragment and/or mutant and/or concatemer
QAGSTPCNGVE

SEQ ID NO: 11 - protein, 1273 aa, artificial sequence
  Sars CoV 2 spike protein of fragment and/or mutant and/or concatemer
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1198 aa, artificial sequence
  Sars CoV 2 spike protein of fragment and/or mutant and/or concatemer
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 13 - protein, 127 aa, artificial sequence
  Sars CoV 2 spike protein of fragment and/or mutant and/or concatemer
YECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVK

SEQ ID NO: 14 - protein, 17 aa, artificial sequence
  Sars CoV 2 spike protein of fragment and/or mutant and/or concatemer
LPDPSKPSKASFIEDLL

SEQ ID NO: 15 - protein, 18 aa, artificial sequence
  Sars CoV 2 spike protein of fragment and/or mutant and/or concatemer
QTNSPRRAASVASQSIIA

SEQ ID NO: 16 - DNA, 3816 bp, artificial sequence
  Sars CoV 2 spike protein of fragment and/or mutant and/or concatemer
atgctgctggtcaaccagtctcaccagggtttcaacaaagaacacacctccaagatggtgtccgccatcgtgctgtacgtgctgttggctgctgctgctcactccgctttcgctgctgactgcgtgaacctgaccaccaggactcaactgcctccagcttacaccaacagcttcacccgtggtgtctactaccccgacaaggtgttccgttcctccgtgctgcactctacccaggacctgttcctgcctttcttctccaacgtgacctggttccacgctatccacgtgtccggtactaacggcaccaagcgtttcgacaaccccgtgctgcctttcaacgacggcgtgtacttcgcttccaccgagaagtccaacatcatccgtggctggatcttcggtactaccctggactccaagactcagtccctgctgatcgtgaacaacgctaccaacgtggtcatcaaagtgtgcgagttccagttctgcaacgaccccttcctgggcgtttactaccacaagaacaacaagtcctggatggaatccgagttccgtgtgtactcctccgctaacaactgcaccttcgagtacgtgtcccagcctttcctgatggacctcgagggcaagcagggcaacttcaagaacctgcgcgagttcgtgttcaagaacatcgacggctacttcaagatctactccaagcacacccctatcaacctcgtgcgtgacctgcctcagggcttctctgctctggaacctctggtggacctgccaatcggtatcaacatcacccgtttccagactctgctggctctgcaccgttcctacttgacccctggcgactcctcttctggatggactgctggtgctgctgcctactacgtgggttacctgcagcctcgtaccttcctgctgaagtacaacgagaacggaaccatcaccgacgctgtggactgcgctctggaccctttgtccgagactaagtgcaccctgaagtccttcaccgtcgagaagggcatctaccagacctccaacttccgtgtgcagcccaccgagtccatcgtgcgtttccctaacatcaccaacttgtgccccttcggcgaggtgttcaacgctactcgtttcgcttccgtgtacgcttggaaccgcaagcgcatctctaactgcgtggccgactactccgtcctgtacaactccgcttccttcagcaccttcaagtgctacggtgtctcccctaccaagctgaacgacctgtgcttcaccaacgtctacgctgactccttcgtgatccgtggcgacgaagtgcgtcagatcgctcctggtcaaaccggcaagatcgctgactacaactacaagctgcccgacgacttcaccggttgcgtgatcgcctggaactccaacaacctggactctaaagtcggcggcaactacaattacctgtaccgtctgttccgcaagtccaacctgaagcctttcgagcgtgacatctctaccgagatctaccaggctggttctaccccttgcaacggtgtcgagggcttcaactgctacttcccactgcagtcctacggtttccagcctaccaacggcgtcggttaccagccttaccgtgtggtggtgctgtccttcgaactgctgcacgctcctgctactgtgtgcggtcccaagaaatccaccaacctggtcaagaacaaatgcgtgaacttcaacttcaacggcctgaccggcaccggtgtcctgaccgagtctaacaagaagttcctgccattccagcaattcggccgtgatatcgctgacaccactgacgctgtgcgcgaccctcagactctggaaatcctggacatcaccccatgcagcttcggtggtgtctccgtgatcacccctggtactaacacctctaaccaggtggccgttctgtaccaggacgtgaactgcaccgaagtgcccgtggctattcacgctgaccagctgactccaacctggcgcgtgtactccaccggttccaacgtgttccagacacgtgctggttgcctgatcggtgctgagcacgtcaacaactcctacgagtgcgacatccccatcggcgctggtatctgcgcttcctaccagactcagaccaactctcctgctggcgctggttccgtggcttcccagtccatcattgcttacactatgtccctgggtgctgagaactccgtcgcttactctaacaactctatcgctatccccaccaacttcaccatctccgtcaccaccgagatcctgcctgtgtccatgaccaagacctccgtggactgcaccatgtacatctgcggcgactccaccgagtgctctaacctgctgttgcagtacggttccttctgcacccagctgaaccgtgctctgaccggtatcgctgtcgagcaggacaagaacacccaagaggtgttcgctcaagtgaagcagatctacaagacccctcctatcaaggacttcggcggtttcaacttctcccagatcttgcccgatccttctaagccctccaagcgctccttcatcgaggatctgctgtttaacaaagtgaccctggctgacgctggcttcatcaagcaatacggcgactgcctgggcgacattgctgctcgtgatctgatctgcgctcagaagttcaacggattgaccgtcctgcctcctctgctgaccgacgagatgatcgctcagtacacctccgctctgctcgctggcactatcacctccggatggactttcggagctggtgccgctctgcagatccccttcgctatgcagatggcttaccgtttcaacggtatcggtgtcacccagaacgtgctctacgagaaccagaagctgatcgccaaccagttcaactctgccatcggaaagatccaggattccctgtcctccaccgcttccgctctgggaaagctgcaagacgtcgtgaaccagaacgctcaggctctgaacaccctcgtgaagcagctgtcctctaacttcggcgctatctcctctgtcctgaacgacatcctgtccaggctggaccctcctgaggctgaagtgcaaatcgaccgtctgatcaccggtcgtctgcagtctctgcagacctacgtgacccagcagttgatccgcgctgctgagatccgtgcttccgctaacctggctgctaccaagatgtccgagtgcgtgctgggacagtccaagcgtgttgacttctgcggcaagggttaccacctgatgagcttccctcagtccgctcctcacggtgtcgtgttcttgcacgtcacctacgtgcccgctcaagagaagaacttcactaccgctccagctatctgccacgacggcaaggctcatttcccacgtgaaggcgtgttcgtgtccaacggcacccattggttcgtcacccagcgcaacttctacgagccccagatcatcactaccgacaacaccttcgtgtccggcaactgcgacgtcgtcatcggtatcgttaacaatactgtgtacgaccctctgcagcccgagctggactccttcaaagaggaactggacaagtacttcaaaaaccacactagccccgacgtggacctgggagacatctctggtatcaacgcttccgtcgtcaacatccagaaagagatcgaccgcctgaacgaggtggccaagaacctcaacgagtccctgatcgacctgcaagagctgggcaaatacgagcagtacatcaagtggcccggttacatccccgaggctcctcgtgatggtcaagcttacgtgcgcaaggacggcgagtgggtgctgttgtctactttcctgcaccaccatcaccaccac

SEQ ID NO: 17 - protein, 1272 aa, artificial sequence
  Sars CoV 2 spike protein of fragment and/or mutant and/or concatemer
MLLVNQSHQGFNKEHTSKMVSAIVLYVLLAAAAHSAFAADCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLHHHHHH

SEQ ID NO: 18 - DNA, 2151 bp, artificial sequence
  Sars CoV 2 spike protein of fragment and/or mutant and/or concatemer
atgctgctggtcaaccagtctcaccagggtttcaacaaagaacacacctccaagatggtgtccgccatcgtgctgtacgtgctgttggctgctgctgctcactccgctttcgctgctgactgcgtgaacctgaccaccaggactcaactgcctccagcttacaccaacagcttcacccgtggtgtctactaccccgacaaggtgttccgttcctccgtgctgcactctacccaggacctgttcctgcctttcttctccaacgtgacctggttccacgctatccacgtgtccggtactaacggcaccaagcgtttcgacaaccccgtgctgcctttcaacgacggcgtgtacttcgcttccaccgagaagtccaacatcatccgtggctggatcttcggtactaccctggactccaagactcagtccctgctgatcgtgaacaacgctaccaacgtggtcatcaaagtgtgcgagttccagttctgcaacgaccccttcctgggcgtttactaccacaagaacaacaagtcctggatggaatccgagttccgtgtgtactcctccgctaacaactgcaccttcgagtacgtgtcccagcctttcctgatggacctcgagggcaagcagggcaacttcaagaacctgcgcgagttcgtgttcaagaacatcgacggctacttcaagatctactccaagcacacccctatcaacctcgtgcgtgacctgcctcagggcttctctgctctggaacctctggtggacctgccaatcggtatcaacatcacccgtttccagactctgctggctctgcaccgttcctacttgacccctggcgactcctcttctggatggactgctggtgctgctgcctactacgtgggttacctgcagcctcgtaccttcctgctgaagtacaacgagaacggaaccatcaccgacgctgtggactgcgctctggaccctttgtccgagactaagtgcaccctgaagtccttcaccgtcgagaagggcatctaccagacctccaacttccgtgtgcagcccaccgagtccatcgtgcgtttccctaacatcaccaacttgtgccccttcggcgaggtgttcaacgctactcgtttcgcttccgtgtacgcttggaaccgcaagcgcatctctaactgcgtggccgactactccgtcctgtacaactccgcttccttcagcaccttcaagtgctacggtgtctcccctaccaagctgaacgacctgtgcttcaccaacgtctacgctgactccttcgtgatccgtggcgacgaagtgcgtcagatcgctcctggtcaaaccggcaagatcgctgactacaactacaagctgcccgacgacttcaccggttgcgtgatcgcctggaactccaacaacctggactctaaagtcggcggcaactacaattacctgtaccgtctgttccgcaagtccaacctgaagcctttcgagcgtgacatctctaccgagatctaccaggctggttctaccccttgcaacggtgtcgagggcttcaactgctacttcccactgcagtcctacggtttccagcctaccaacggcgtcggttaccagccttaccgtgtggtggtgctgtccttcgaactgctgcacgctcctgctactgtgtgcggtcccaagaaatccaccaacctggtcaagaacaaatgcgtgaacttcaacttcaacggcctgaccggcaccggtgtcctgaccgagtctaacaagaagttcctgccattccagcaattcggccgtgatatcgctgacaccactgacgctgtgcgcgaccctcagactctggaaatcctggacatcaccccatgcagcttcggtggtgtctccgtgatcacccctggtactaacacctctaaccaggtggccgttctgtaccaggacgtgaactgcaccgaagtgcccgtggctattcacgctgaccagctgactccaacctggcgcgtgtactccaccggttccaacgtgttccagacacgtgctggttgcctgatcggtgctgagcacgtcaacaactcctacgagtgcgacatccccatcggcgctggtatctgcgcttcctaccagactcagaccaactctcctgctggcgctggtcaccaccatcaccaccac

SEQ ID NO: 19 - protein, 717 aa, artificial sequence
  Sars CoV 2 spike protein of fragment and/or mutant and/or concatemer
MLLVNQSHQGFNKEHTSKMVSAIVLYVLLAAAAHSAFAADCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGHHHHHH

SEQ ID NO: 20 - DNA, 807 bp, artificial sequence
  Sars CoV 2 spike protein of fragment and/or mutant and/or concatemer
atgctgctggtcaaccagtctcaccagggtttcaacaaagaacacacctccaagatggtgtccgccatcgtgctgtacgtgctgttggctgctgctgctcactccgctttcgctgctgatcgtgtgcagcctaccgagtccatcgtgcgtttccccaacatcaccaacttgtgccccttcggcgaggtgttcaacgctacccgtttcgcttccgtgtacgcttggaaccgcaagcgcatctctaactgcgtggccgactactccgtcctgtacaactccgcttccttcagcaccttcaagtgctacggtgtctcccctaccaagctgaacgacctgtgcttcaccaacgtctacgctgactccttcgtgatccgtggcgacgaagtgcgtcagatcgctcctggtcaaaccggcaagatcgctgactacaactacaagctgcccgacgacttcaccggttgcgtgatcgcctggaactccaacaacctggactccaaagtcggtggcaactacaattacctgtaccgcctgttccgcaagtccaacctgaagcctttcgagcgtgacatctccaccgagatctaccaggctggttctaccccttgcaacggtgtcgagggcttcaactgctacttcccactgcagtcctacggtttccagcctaccaacggcgtgggttaccagccttaccgtgtggtggtgctgtccttcgaactgctgcacgctcctgctactgtgtgcggtcccaagaaatccaccaacctggtcaagaacaaatgcgtgaacttccaccaccaccatcaccac

SEQ ID NO: 21 - protein, 269 aa, artificial sequence
  Sars CoV 2 spike protein of fragment and/or mutant and/or concatemer
MLLVNQSHQGFNKEHTSKMVSAIVLYVLLAAAAHSAFAADRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 22 - DNA, 957 bp, artificial sequence
  Sars CoV 2 spike protein of fragment and/or mutant and/or concatemer
atgctgctggtcaaccagtctcaccagggtttcaacaaagaacacacctccaagatggtgtccgccatcgtgctgtacgtgctgttggctgctgctgctcactccgctttcgctgctgatcgtgtgcagcctaccgagtccatcgtgcgtttccccaacatcaccaacttgtgccccttcggcgaggtgttcaacgctacccgtttcgcttccgtgtacgcttggaaccgcaagcgcatctctaactgcgtggccgactactccgtcctgtacaactccgcttccttcagcaccttcaagtgctacggtgtctcccctaccaagctgaacgacctgtgcttcaccaacgtctacgctgactccttcgtgatccgtggcgacgaagtgcgtcagatcgctcctggtcaaaccggcaagatcgctgactacaactacaagctgcccgacgacttcaccggttgcgtgatcgcctggaactccaacaacctggactccaaagtcggtggcaactacaattacctgtaccgcctgttccgcaagtccaacctgaagcctttcgagcgtgacatctccaccgagatctaccaggctggttctaccccttgcaacggtgtcgagggcttcaactgctacttcccactgcagtcctacggtttccagcctaccaacggcgtgggttaccagccttaccgtgtggtggtgctgtccttcgaactgctgcacgctcctgctactgtgtgcggtcccaagaaatccaccaacctggtcaagaacaaatgcgtgaacttcaacttcaacggcctgaccggcaccggtgtcctgaccgagtctaacaagaagttcctgcctttccagcaattcggccgtgatatcgctgacaccaccgacgctgtgcgtgaccctcagactctggaaatcctggacatcaccccttgctctcaccaccaccatcaccac

SEQ ID NO: 23 - protein, 319 aa, artificial sequence
  Sars CoV 2 spike protein of fragment and/or mutant and/or concatemer
MLLVNQSHQGFNKEHTSKMVSAIVLYVLLAAAAHSAFAADRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSHHHHHH

SEQ ID NO: 24 - DNA, 855 bp, artificial sequence
  Sars CoV 2 spike protein of fragment and/or mutant and/or concatemer
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagaaacatcaccaacttgtgccccttcggcgaggtgttcaacgctactcgtttcgcttccgtgtacgcttggaaccgcaagcgcatctctaactgcgtggccgactactccgtcctgtacaactccgcttccttcagcaccttcaagtgctacggtgtctcccctaccaagctgaacgacctgtgcttcaccaacgtctacgctgactccttcgtgatccgtggcgacgaagtgcgtcagatcgctcctggtcaaaccggcaagatcgctgactacaactacaagctgcccgacgacttcaccggttgcgtgatcgcctggaactccaacaacctggactctaaagtcggcggcaactacaattacctgtaccgtctgttccgcaagtccaacctgaagcctttcgagcgtgacatctctaccgagatctaccaggctggttctaccccttgcaacggtgtcgagggcttcaactgctacttcccactgcagtcctacggtttccagcctaccaacggcgtcggttaccagccttaccgtgtggtggtgctgtccttcgaactgctgcacgctcctgctactgtgcaccaccatcaccaccac

SEQ ID NO: 25 - protein, 285 aa, artificial sequence
  Sars CoV 2 spike protein of fragment and/or mutant and/or concatemer
MRFPSIFTAVLFAASSALAAPVNTTTEDETAQIPAEAVIGYSDLEGDFDVAVLPFSNSTNNGLLFINTTIASIAAKEEGVSLEKRNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVHHHHHH

SEQ ID NO: 26 - protein, 1273 aa, artificial sequence
  Sars CoV 2 spike protein of fragment and/or mutant and/or concatemer
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1198 aa, artificial sequence
  Sars CoV 2 spike protein of fragment and/or mutant and/or concatemer
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 28 - protein, 1273 aa, artificial sequence
  Sars CoV 2 spike protein of fragment and/or mutant and/or concatemer
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - protein, 1198 aa, artificial sequence
  Sars CoV 2 spike protein of fragment and/or mutant and/or concatemer
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 30 - protein, 344 aa, artificial sequence
  Sars CoV 2 spike protein of fragment and/or mutant and/or concatemer
QAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPGSGSQAGSTPCNGVEGFNCYFPLQSYGFQPTNGVGYQPHHHHHH

SEQ ID NO: 31 - protein, 302 aa, artificial sequence
  Sars CoV 2 spike protein of fragment and/or mutant and/or concatemer
QAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEGSGSQAGSTPGNGVEHHHHHH

SEQ ID NO: 32 - protein, 324 aa, artificial sequence
  Sars CoV 2 spike protein of fragment and/or mutant and/or concatemer
QPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPGSGSQPTNGVGYQPHHHHHH

SEQ ID NO: 33 - protein, 333 aa, artificial sequence
  Sars CoV 2 spike protein of fragment and/or mutant and/or concatemer
GFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGSGSGFNGYFPLQSYGHHHHHH

SEQ ID NO: 34 - protein, 322 aa, artificial sequence
  Sars CoV 2 spike protein of fragment and/or mutant and/or concatemer
SNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYGSGSSNNLDSKVGGNYHHHHHH

SEQ ID NO: 35 - protein, 302 aa, artificial sequence
  Sars CoV 2 spike protein of fragment and/or mutant and/or concatemer
QTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIGSGSQTNSPRRARSVASQSIHHHHHH

SEQ ID NO: 36 - protein, 34 aa, artificial sequence
  Sars CoV 2 spike protein of fragment and/or mutant and/or concatemer
QAGSTPCNGVEGFNCYFPLQSYGFQPTNGVGYQP

SEQ ID NO: 37 - protein, 11 aa, artificial sequence
  Sars CoV 2 spike protein of fragment and/or mutant and/or concatemer
QAGSTPGNGVE

SEQ ID NO: 38 - protein, 10 aa, artificial sequence
  Sars CoV 2 spike protein of fragment and/or mutant and/or concatemer
QPTNGVGYQP

SEQ ID NO: 39 - protein, 12 aa, artificial sequence
  Sars CoV 2 spike protein of fragment and/or mutant and/or concatemer
GFNGYFPLQSYG

SEQ ID NO: 40 - protein, 12 aa, artificial sequence
  Sars CoV 2 spike protein of fragment and/or mutant and/or concatemer
SNNLDSKVGGNY

SEQ ID NO: 41 - protein, 16 aa, artificial sequence
  Sars CoV 2 spike protein of fragment and/or mutant and/or concatemer
QTNSPRRARSVASQSI

(19) United States
(12) Patent Application Publication
(10) Pub. No.: US 2023/0192813 A1
(43) Pub. Date: Jun. 22, 2023

(54) ANTIBODY THAT BINDS SPECIFICALLY TO THE SARS COV 2 SPIKE PROTEIN, AND METHODS FOR ITS MANUFACTURE

(71) Applicant: IGNOVA GMBH, Visbek (DE)

(72) Inventors: Jan WESJOHANN, Visbek (DE); Michael GERRITS, Osnabrück (DE); Tonchy UGRINOVIC, Oldenburg (DE); Nguyen VAN SA, Gifu-City (JP); Andreas MICHELS, Düsseldorf (DE)

(21) Appl. No.: 17/926,359

(22) PCT Filed: May 18, 2021
(86) PCT No.: PCT/EP2021/063169

§ 371 Date: Nov. 18, 2022

Related U.S. Application Data
(60) Provisional application No. 63/026,188, filed on May 18, 2020.

(30) Foreign Application Priority Data
May 18, 2020 (EP) 20175218.5

(51) Int. Cl. C07K 16/02; C07K 16/04; C07K 16/10; A61P 31/14; A61K 45/06; A61K 39/42

(52) U.S. Cl. CPC C07K 16/02; C07K 16/04; C07K 16/10; A61P 31/14; A61K 45/06; A61K 39/42; C07K 2317/76; C07K 2317/12; C07K 2317/23

(57) ABSTRACT

The present invention relates to an antibody that binds specifically to the SARS CoV-2 spike protein, and methods for its manufacture.

### FIELD OF THE INVENTION

The present application relates to antibodies that bind specifically to the SARS CoV 2 spike protein, and methods for its manufacture.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. The incorporation is primarily meant for enablement purposes. In the event that there are any inconsistencies between the teachings of one or more of the references incorporated herein and the present disclosure, the teachings of the present specification are intended.

### BACKGROUND

Severe acute respiratory syndrome coronavirus 2 (SARS-CoV-2) is the strain of coronavirus that causes coronavirus disease 2019 (COVID-19), a respiratory illness. Colloquially known as coronavirus, it was previously referred to by its provisional name 2019 novel coronavirus (2019-nCoV). As described by the National Institutes of Health, it is the successor to SARS-CoV-1. SARS-CoV-2 is a positive-sense single-stranded RNA virus. It is contagious in humans, and the World Health Organization (WHO) has designated the ongoing pandemic of COVID-19 a Public Health Emergency of International Concern.

Taxonomically, SARS-CoV-2 is a strain of severe acute respiratory syndrome-related coronavirus (SARSr-CoV). It is believed to have zoonotic origins and has close genetic similarity to bat coronaviruses, suggesting it emerged from a bat-borne virus. The virus shows little genetic diversity, indicating that the spillover event introducing SARS-CoV-2 to humans is likely to have occurred in late 2019.

Based on the low variability exhibited among known SARS-CoV-2 genomic sequences, the strain is thought to have been detected by health authorities within weeks of its emergence among the human population in late 2019. The earliest case of infection currently known is date back to 17 Nov. 2019 or possibly 1 Dec. 2019. The virus subsequently spread to all provinces of China and to more than 150 other countries in Asia, Europe, North America, South America, Africa, and Oceania. Human-to-human transmission of the virus has been confirmed in all these regions. On 30 Jan. 2020, SARS-CoV-2 was designated a Public Health Emergency of International Concern by the WHO, and on 11 Mar. 2020 the WHO declared it a pandemic.

The basic reproduction number R of the virus has been estimated to be between 1.4 and 3.9. This means each infection from the virus is expected to result in 1.4 to 3.9 new infections when no members of the community are immune and no preventive measures are taken. The reproduction number may be higher in densely populated conditions such as those found on cruise ships

The therapeutic options to treat COVID-19 disease or prevent it's outbreak are limited. Likewise, besides measures like social distancing and using protective gear, options to avoid SARS-CoV-2 infection are also limited.

Also, it has been noticed that in many severe cases of COVID-19, the patient's own immune contributes to the fatal etiopatology. For example, after SARS CoV-2 infection, the patient secretes interferons to support the body's virus defense. At a certain point, however, the patient's response may become so strong (“overshoot”) that its effect can be counterproductive. For example, numerous immune cells can enter our lungs and cause the membrane through which oxygen normally passes from the air into the blood to thicken. The exchange of gases is restricted, and in the worst case, ventilation may be necessary. Sometimes the reaction can overshoot and be directed against healthy cells as well

On the basis of the above, it is one object of the present invention to provide methods and compositions that broaden the therapeutic options to treat COVID-19 disease or prevent it's outbreak.

It is one other object of the present invention to provide methods and compositions that broaden the options to avoid SARS-CoV-2 infection.

It is one other object of the present invention to provide methods and compositions that help controlling the overshooting immune response concomitant with severe COVID 19 etiopathology, or that do not result in overshooting immune responses when administered to a patient.

### BRIEF DESCRIPTION OF THE DRAWINGS

Table 1 shows preferred length ranges of the peptides comprising any of SEQ ID NO: 1-SEQ ID NO: 10 that can be used for immunization. Note that the lengths ranges of SEQ ID NOs 1, 2, 5, 6, or 7 apply to their corresponding mutated counterparts, SEQ ID NOs 11, 12, 13, 14 or 15, too.

FIG. 1 shows schematically the generation of IgY antibodies according to the present invention.

FIG. 2 shows the elative distribution of IgG, IgA and IgM in colostrum (outer circle) and in milk (inner circle) of five species. The relative size of the circles represents the overall concentration of total immunoglobulins found among the species and the concentrations in colostrum vs. milk.

FIG. 3 shows a sequence alignment of SEQ ID NOs: 1-10. The sequence motifs of the S1/S2 cleavage site and S2′ are shown in bold underline in SEQ ID NO: 1.

FIG. 4 shows the domain structure of the SARS Cov2 spike protein. The protein consists of Domains S1 and S2. S1 contains the receptor binding domain responsible for binding to ACE2. S2 is responsible for fusion with the host cell. Upon binding to ACE2 a conformational change in Spike is induced and TMPRSS cleaves at S1/S2 and S2′. TM=Transmembrane domain HR-1=heptad repeat 1 (N-positioned); HR 2=heptad repeat 2 (C-positioned); FP=Fusion peptide.

FIG. 5 shows results of a SARS-CoV2 spike protein binding assay.

FIG. 6 shows results of a CPE neutralization assay.

FIG. 7 shows the results of the fluorescent plaque reduction assay.

FIG. 8 shows the concatemers used in Example 6.

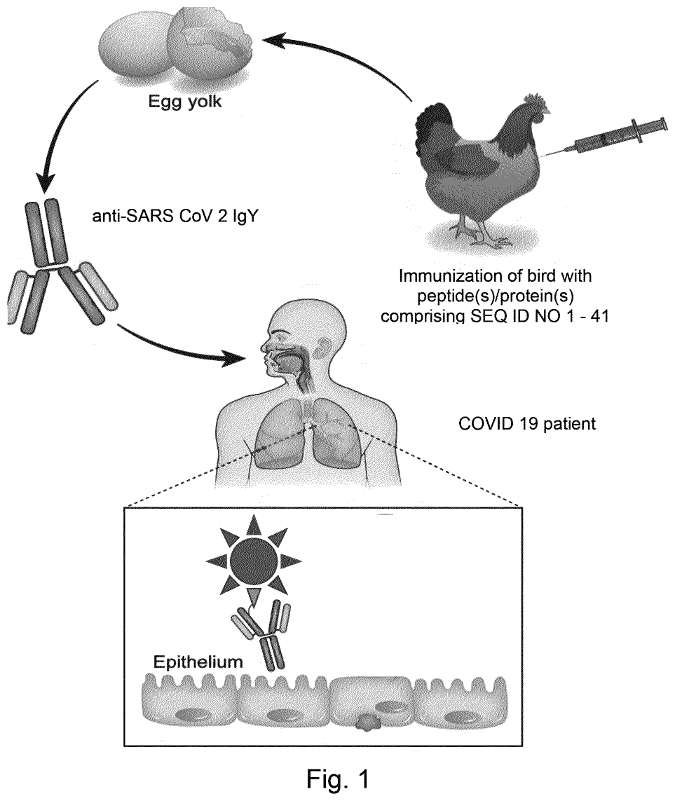

### SUMMARY OF THE INVENTION

The present invention provides, among other things, a method for isolating or producing antibodies against the SARS CoV 2 spike protein. The invention and general advantages of its features will be discussed in detail below.

According to one aspect of the invention, a method for isolating or producing antibodies is provided, the method comprising:
immunizing a female vertebrate animal by administration of at least one protein or peptide comprising at least one amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41,
collecting one or more lactation or ovulation product from the animal after immunization, and
isolating or purifying at least one antibody from the products,

wherein the protein or peptide is, comprises, or is a fragment of, the SARS CoV 2 spike protein.

Note that SEQ ID NOs 1-10 correspond to the wildtype spike protein, or fragments or epitopes thereof. Note that SEQ ID NOs 11-15 comprise one or two substitutions relative to SEQ ID NOs 1, 2, 5, 6 or 7.

In SEQ ID NOs: 11, 12, 13 and 15, the Arginine residue (R) in the peptide motif “ARS” as in the corresponding wildtype protein or peptide has been replaced by an Alanine residue (A), to arrive at the peptide motif “AAS”.

The same applies to SEQ ID NO: 11, 12, and 14, where the Arginine residue (R) in the peptide motif “KRS” as in the corresponding wildtype protein or peptide has been replaced by an Alanine residue (A), to arrive at the peptide motif “KAS”.

The following table provides an overview of the wildtype sequences and their mutated counterparts

| Wildtype | Mutated counterpart | |
|---|---|---|
| SEQ ID NO | SEQ ID NO | Mutation |
| 1 | 11 | ARS −> AAS; KRS −> KAS |
| 2 | 12 | ARS −> AAS; KRS −> KAS |
| 5 | 13 | ARS −> AAS |
| 6 | 14 | KRS −> KAS |
| 7 | 15 | ARS −> AAS |

The peptide motifs AR and KR, as in the corresponding wildtype protein or peptide represent a cleavage site for proteases, like e.g. Furin and/or TMPRSS2 (transmembrane serine protease type 2) and other related proteases. To stabilize the position against proteolytic cleavage, the inventors have mutated the respective sites in the proteins and peptides used for immunization. For explanation of the terms “S1” and “S2” see elsewhere in the text.

The term “peptide”, as used herein, relates to a molecule which consists of 2 or more amino acids bound to one another by a peptide bond.

The term “oligopeptide”, as used herein, relates to a peptide which has a length of between ≥2 and ≤20 amino acids).

The term “polypeptide”, as used herein, relates to a peptide which has a length of length of between >20 and <50 amino acids).

The term “protein”, as used herein, relates to a peptide which has a length of >50 amino acids or comprises two or more peptides associated to one another covalently (e.g., by a linker or a disulfide bridge) or non-covalently.

Severe acute respiratory syndrome coronavirus 2 (SARS-CoV-2) is the virus strain that causes coronavirus disease 2019 (COVID-19), a respiratory illness. It is colloquially known as the coronavirus, and was previously referred to by its provisional name 2019 novel coronavirus (2019-nCoV). SARS-CoV-2 is a positive-sense single-stranded RNA virus. It is contagious in humans.

Taxonomically, SARS-CoV-2 is a strain of severe acute respiratory syndrome-related coronavirus (SARS-CoV). It is believed to have zoonotic origins and has close genetic similarity to bat coronaviruses, suggesting it emerged from a bat-borne virus. An intermediate animal reservoir such as a pangolin is also thought to be involved in its introduction to humans. The virus shows little genetic diversity, indicating that the spillover event introducing SARS-CoV-2 to humans is likely to have occurred in late 2019.

The SARS-CoV-2 virion is approximately 50-200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein, which has been imaged at the atomic level using cryogenic electron microscopy, is the protein responsible for allowing the virus to attach to and fuse with the membrane of a host cell.

The spike protein consists of Domains 51, S2 and S2′ (see FIG. 4). 51 contains the receptor binding domain responsible for binding to ACE2. S2 is responsible for fusion with the host cell. Upon binding to ACE2 a conformational change in Spike is induced and TMPRSS cleaves at S1/S2 and S2′

SARS-CoV-2 spike protein has affinity to the receptor angiotensin converting enzyme 2 (ACE2) on human cells to use them as a mechanism of cell entry. After a SARS-CoV-2 virion attaches to a target cell, the cell's protease TMPRSS2 cuts open the spike protein of the virus, exposing a fusion peptide. The virion then releases RNA into the cell and forces the cell to produce and disseminate copies of the virus, which infect more cells.

The corona spike protein is a class I fusion protein, and forms trimers. Each monomer of trimeric S protein is about 180 kDa. The formation of an α-helical coiled-coil structure is characteristic of this class of fusion protein, which contain in their C-terminal part regions predicted to have an α-helical secondary structure and to form coiled-coils.

S1 contains two subdomains, a N-terminal domain (NTD) and a C-terminal domain (CTD). Both are able to function as receptor binding domains (RBDs) and bind variety of proteins and sugars.

Coronavirus spike proteins contain two heptad repeats in their S2 domain, a feature typical of a class I viral fusion proteins. Heptad repeats comprise a repetitive heptapeptide abcdefg with a and d being hydrophobic residues characteristic of the formation of coiled-coil that participate in the fusion process. For SARS-CoV the post-fusion structures of the HR have been solved; they form the characteristic six-helix bundle.

Studies have shown that SARS-CoV-2 has a higher affinity to human ACE2 than the original SARS virus strain.

The following table shows some of the peptides that are being used for immunization.

| SEQ | | | AA residues |
|---|---|---|---|
| ID | | Length | within SEQ |
| NO | qualifier | (AA) | ID NO: 1 |
| 1 | Full length SARS-CoV-2 Spike protein | 1273 | |
| 2 | Full length S1/S2 spike ectodomain | 1198 | 16 to 1213 |
| 3 | Binding Domain CBD | 273 | 319 to 591 |
| 4 | SHORTER Fragment with binding | 201 | 330 to 530 |
| | domain CBD | | |
| 5 | Protease cleavage domain S1/S2 | 127 | 660 to 786 |
| 6 | S2′proteolytic site epitope | 17 | 806-822 |
| 7 | S1/S2 proteolytic site epitope | 18 | 677-694 |
| 8 | Binding motif loop 1a epitope | 11 | 440 to 448 |
| 9 | Binding motif loop 1b epitope | 10 | 498 to 507 |
| 10 | Binding motif loop 2 epitope | 11 | 474 to 484 |
| 11 | Full length SARS-CoV-2 Spike protein, | 1273 | |
| | with R in the wildtype peptide motif | | |
| | ARS or KRS replaced by A | | |
| 12 | Full length S1/S2 spike ectodomain, | 1198 | 16 to 1213 |
| | with R in the wildtype peptide motif | | |
| | ARS or KRS replaced by A | | |
| 13 | Protease cleavage domain S1/S2, with | 127 | 660 to 786 |
| | R in the wildtype peptide motif ARS | | |
| | or KRS replaced by A | | |
| 14 | S2′proteolytic site epitope, with | 17 | 806-822 |
| | R in the wildtype peptide motif ARS | | |
| | or KRS replaced by A | | |
| 15 | S1/S2 proteolytic site epitope, with | 18 | 677-694 |
| | R in the wildtype peptide motif ARS | | |
| | or KRS replaced by A | | |

Out of these sequences, SEQ ID NO: 1 comprises the transmembrane part of the spike domain, whereas the other sequences comprise only parts of the ectodomain devoid of the transmembrane domain.

Some of the peptides comprise sequences which in the spike protein are functionally relevant for virus infection (e.g., binding to ACE2, or being primed by protease).

The method according to the invention has significant advantages in particular when it comes to respond on global pandemia like COVID 19. With this method, antibodies can be produced in high numbers at high speed, so a quick response is possible.

Furthermore, it does not require the use of cell cultures not time-consuming individual immunization of eggs, Once immunized a female bird can produce large numbers of antibody comprising eggs, and by repeated immunization the production can be maintained over long stretches of time.

In one embodiment, the antibody isolated from the lactation or ovulation products is a polyclonal antibody.

According to one embodiment of the method according to the invention, the animal is a female bird.

According to one embodiment of the method according to the invention the animal is an anseriform bird.

According to one embodiment of the method according to the invention, the animal is a chicken.

According to one embodiment of the method according to the invention, the ovulation product that is collected from the animal is one or more eggs.

According to one embodiment of the method according to the invention, the antibody that is purified from the eggs is IgY.

Immunoglobulin Y (abbreviated as IgY) is a type of immunoglobulin which is the major antibody in bird, reptile, and lungfish blood. It is also found in high concentrations in chicken egg yolk. As with the other immunoglobulins, IgY is a class of proteins which are formed by the immune system in reaction to certain foreign substances, and specifically recognize them.

IgY differs both structurally and functionally from mammalian IgG, and does not cross-react with antibodies raised against mammalian IgG.

The yolk of an immunised bird's egg contains a high concentration of IgY, so as to immunize the offspring against the respective pathogen. Egg yolk is a complex mixture of water (50%) lipids (32-35%) and proteins (16%). Proteins residing within the yolk are of 4 types: lipovitellins, phosphorous-containing lipoproteins (40%), apovitellenins, containing less phosphorous but more lipid (37.3%), phosvitin, a phosphoprotein (13.4%), and the livetins (9.3%), of which IgY is a part. Removal of the yolk lipids and lipoproteins leaves a water soluble fraction, containing IgY along with other proteins, which crudely could be compared to an animal serum, in terms of usability in immunoassays.

Another advantage is that IgY do not bind to Fc gamma receptors of the human immune system. As a consequence, they cannot bind complement or be picked up by macrophages, meaning that their role in antiviral treatment will be reduced to mere inhibition of the cell entry. Hence, the risk of overshooting immune responses—a symptom that is often fatal in COVID 19 patients—is reduced.

According to one embodiment of the method according to the invention, the animal is a female mammal.

According to one embodiment of the method according to the invention, the animal is a ruminant.

According to one embodiment of the method according to the invention, the animal is a cow, pig, camel, horse, donkey, goat or sheep.

According to one embodiment of the method according to the invention, the lactation product collected from the animal is, whey, milk or colostrum.

According to one embodiment of the method according to the invention, the antibody is at least one of IgA, IgG, and IgM.

For mammalian offspring, the immunoglobulins comprised in mammalian milk, whey and colostrum provide the major antimicrobial protection against microbial infections, and thus confer a passive immunity to the newborn until its own immune system matures.

The concentration in milk and colostrum of specific antibodies against pathogens can be raised by immunizing female mammals, like cows, with these pathogens or their antigens.

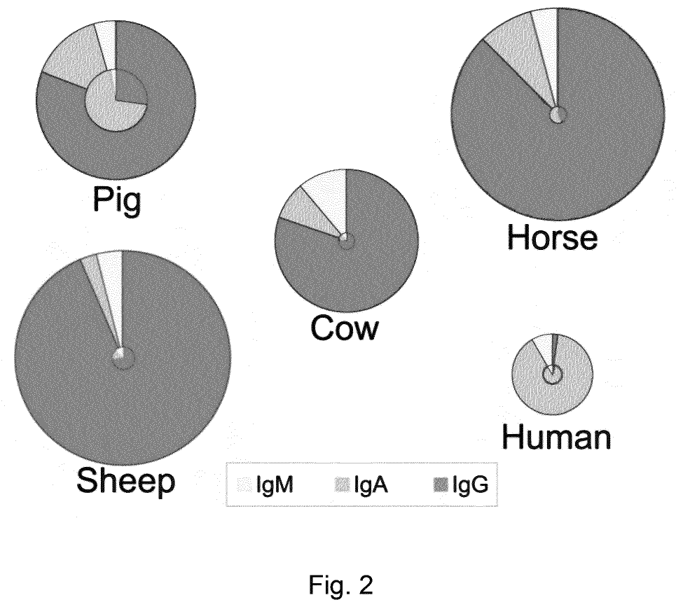

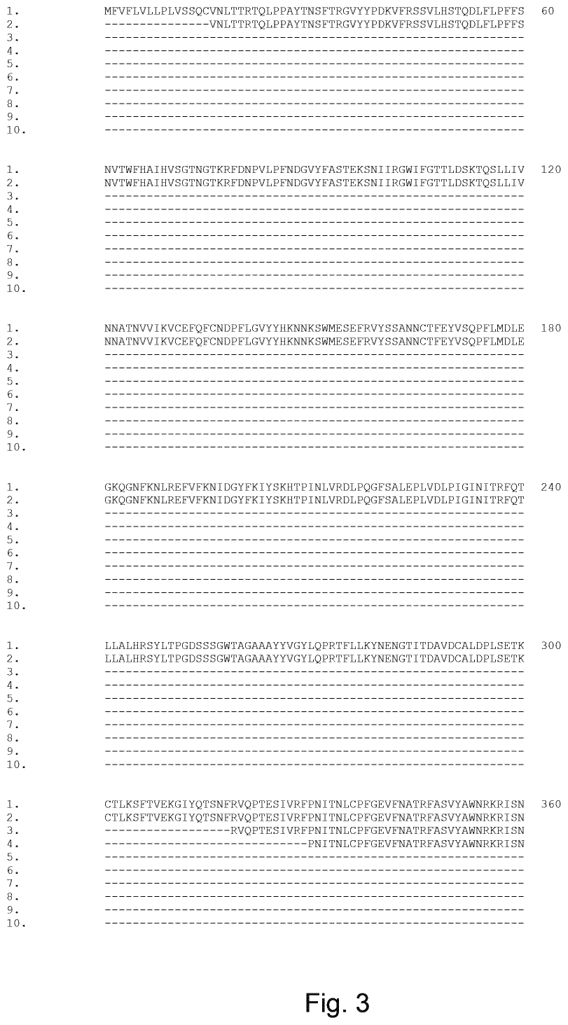

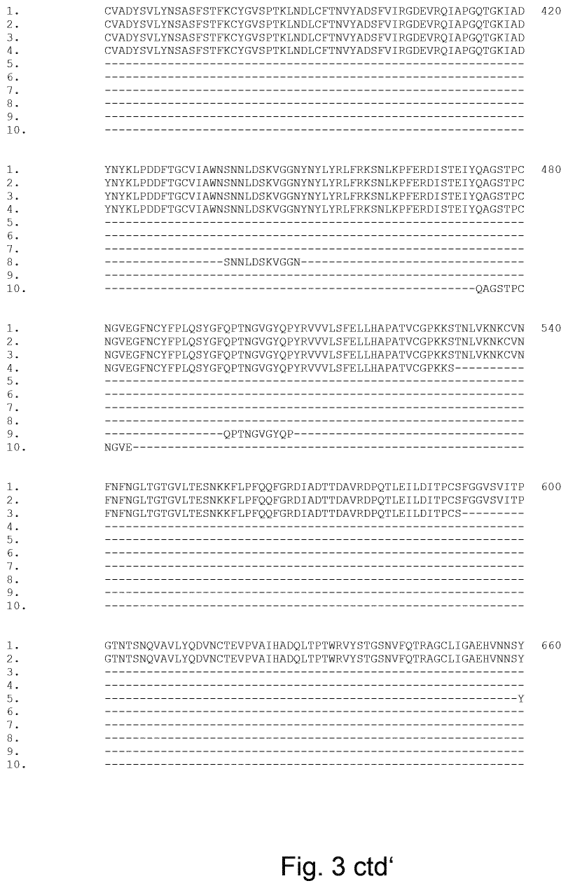

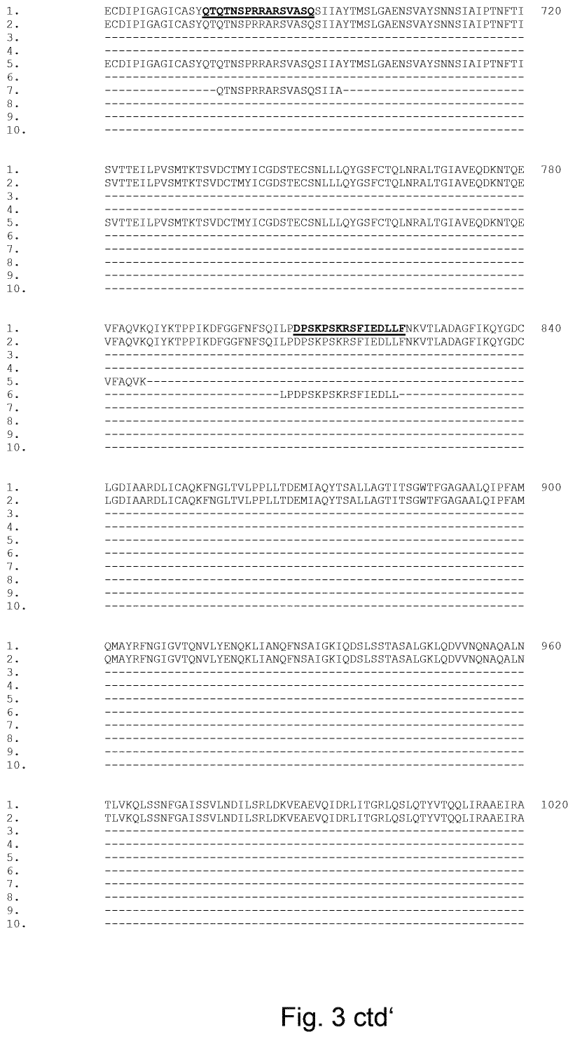

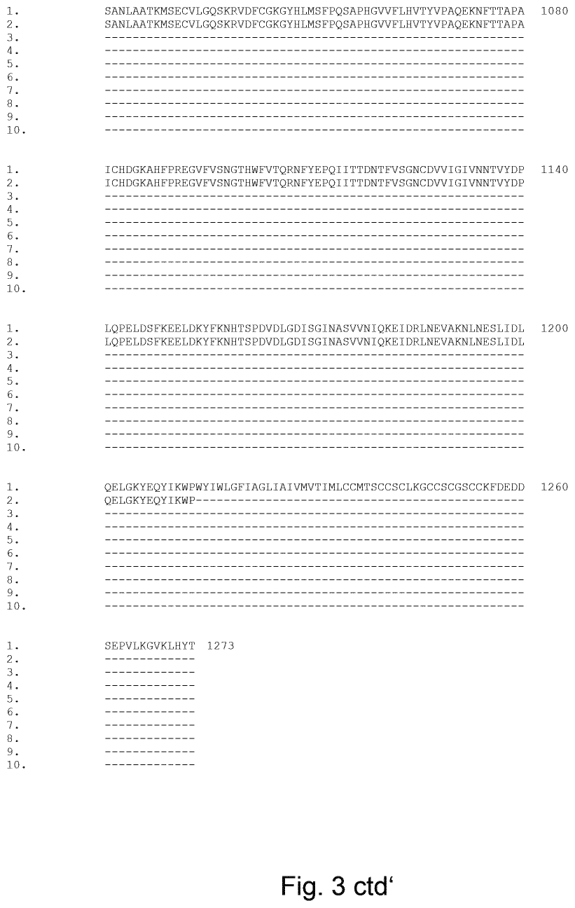

FIG. 2 shows the relative distribution of IgG, IgA and IgM in colostrum (outer circle) and in milk (inner circle) of five mammalian species. The relative size of the circles represents the overall concentration of total immunoglobulins found among the species and the concentrations in colostrum vs. milk.

According to one embodiment of the method according to the invention, the protein or peptide comprising at least one amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41 has a maximum length as set forth in table 1. Note that the lengths ranges of SEQ ID NOs 1, 2, 5, 6, or 7 apply to their corresponding mutated counterparts, SEQ ID NOs 11, 12, 13, 14 or 15, too.

According to one embodiment of the method according to the invention, two or more proteins or peptides comprising at least one amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41 are used for immunizing the animal.

In several embodiments, the following combinations are preferred for immunizing the animal:
(1) at least one protein or peptide comprising at least one amino acid sequence selected from the group consisting of SEQ ID NO: 3-SEQ ID NO: 5 and SEQ ID NO 15 and at least one protein or peptide comprising at least one amino acid sequence selected from the group consisting of SEQ ID NO: 6-SEQ ID NO: 10 and SEQ ID NO: 14-SEQ ID NO: 15,
(2) at least one protein or peptide comprising at least one amino acid sequence selected from the group consisting of SEQ ID NO: 1-SEQ ID NO: 3 and SEQ ID NO: 11-SEQ ID NO: 12,
(3) and at least one protein or peptide comprising at least one amino acid sequence selected from the group consisting of SEQ ID NO: 4-SEQ ID NO: 10 and SEQ ID NO: 13-SEQ ID NO: 15,

Such use of two or more proteins or peptides can comprise simultaneous administration of the different proteins or peptides, or subsequent administration of the different proteins or peptides.

simultaneous administration, as used herein, means that the two peptides are administered at roughly the same time, in the same dosage unit or in different dosage units, but with an interval of ≤3 hrs, ≤2 hrs, ≤1 hrs or ≤30 mins.

Subsequent administration, as used herein, means that the two peptides are administered at different points of time, with an interval of >3 hrs, >10 hrs, >24 hrs, >2d, >4d, >7d, >2 wks or >4 wks.

According to one embodiment of the method according to the invention,
a) at least one single chain protein or peptide is used for immunizing which comprises two or more subsequences each comprising an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, and
b) at least one homo- or heterodimer, -oligomer or -multimer is used for immunizing which comprises two or more chains each comprising an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO 29.

As regards option a), the two or more subsequences can be identical (i.e., comprising amino acid sequences having the same SEQ ID NO), or different (i.e., comprising amino acid sequences having different SEQ ID NOs).

Such single chain protein or peptide which comprises two or more subsequences is preferably a concatemer as described herein elsewhere,

A concatemer is a peptide or protein molecule that contains multiple copies of the same peptide sequence linked in series, either directly or by a linker, e.g., a GS linker.

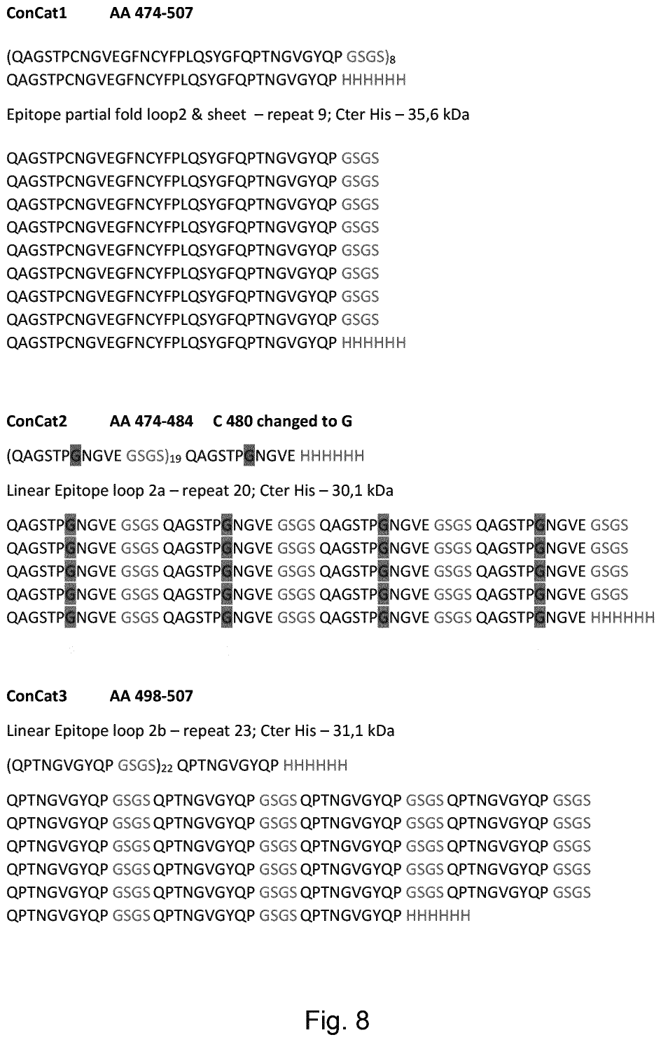

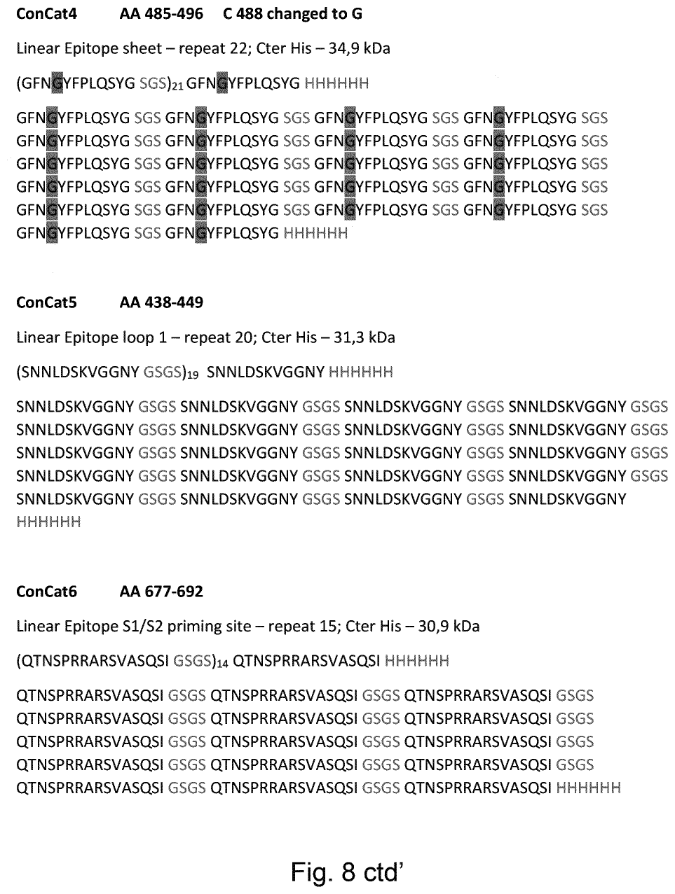

Such single chain protein or peptide which comprises preferably an amino acid sequence according to any one of SEQ ID NO 30-35. These concatemers are shown in FIG. 8, as well as their subsequences (“building clocks”), which are disclosed as SEQ ID NO 36-41.

The concatemers have been chosen to comprise repetitions of epitopes binding of which by an antibody is deemed to have an inhibitory effect of virus propagation. By using several repeats of these epitopes a critical mass is achieved which renders the concatemer immunogenic, so that a significant immune response can be reached.

It is important to mention that the said concatemers can be linear and/or denaturated peptides preoteins (for this reason, as shown in the sequence table herein, in some building blocks Cys has been substutured by Gly, to avoid the formation of disulfide bridges.

In contrast thereto, the peptides and proteins according to any one of SEQ ID NO 1-5, SEQ ID NO 11-13, or SEQ ID NO 17-29 are preferably fully folded proteins.

It is further important to understand that, beyond one of the general concepts disclosed herein, which is a method for isolating or producing antibodies in a lactation or ovulation product of a female vertebrate, the embodiments in which concatemers as described herein, in particular the concatemers according to any one of SEQ ID NO 30-35 and/or the subsequences (“building clocks”) disclosed as SEQ ID NO 36-41 are provided, can be used also for direct immunization of animals or humans, e.g., in order to generate immunity against the SARS CoV 2 virus, in order to create monoclonal antibodies by the hybridoma technique, or in order to vaccinate a human or animal. The advantages and preferred embodiment discussed herein elsewhere translate also to such applications.

As regards option b), the two or more chains can be identical (i.e., comprising amino acid sequences having the same SEQ ID NO), or different (i.e., comprising amino acid sequences having different SEQ ID NOs). The two or more chains can be linked to one another by one or more peptide linkers, or by one or more disulfide bridges.

According to one embodiment of the method according to the invention, at least one protein or peptide used for immunizing is conjugated to a second molecule known to be immunogenic in the animal being immunized.

In such way, a potential problem caused by potentially insufficient length of the protein or peptide, resulting in insufficient immunogenicity, is addressed. It is hence particularly useful if the length of the protein or peptide is not immunogenic enough.

In one embodiment, such second molecule is a protein (also called “carrier protein”). Examples of such carrier proteins include but are not limited to keyhole limpet hemocyanin, serum albumin, bovine thyroglobulin, and soybean trypsin inhibitor.

According to one embodiment of the method according to the invention, the isolation or purification of the antibody comprises the steps of
a) removal of the bulk of the lipids and lipoproteins from the yolk (“delipidation”), and
b) concentration or purification of the IgY fraction.

A number of methods which remove most of the lipids/lipoproteins are shown in the following:
Polyethylene Glycol precipitation
Dextran Sulphate Precipitation
Organic Solvent Lipid Solubilization
Natural Gums, Xantham/Carageenan Precipitation
Lipid Dilution/Ultrafiltration
Phosphate:Triton X delipidation

Following the delipidation of the egg yolk, the almost lipid-free solution can then be treated in a number of ways to concentrate/purify the IgY fraction:
Polyethylene Glycol Precipitation
Sodium Sulphate Precipitation
Ultrafiltration/Ammonium Sulphate Precipitation
Preparative Electrophoresis
Sodium chloride precipitation

These methods have been reviewed in Kovacs-Nolan and Mine (2004), and Akita and Nakai (1993) the contents of which are incorporated herein by reference. Other methods are disclosed in Tan et al (2001), Hodek et al (2013) and Stålberg and Larsson (2001), the contents of which are incorporated herein by reference.

There are a number of IgY Purification Kits commercially available that use one or more of the methods described above:

| | IgY | IgY Yield | IgY Purity | Cost |
|---|---|---|---|---|
| | Purification | (mg IgY/gram | (% Purity by | (per mg IgY |
| Manufacturer | Kit Name | yolk) | SDS PAGE) | purified) |
| Gallus Immunotech | IgY Eggspress | 4-9 | 85-90 | a |
| | Purification Kit | | | |
| Pierce Biotechnology/ | Chicken IgY | 4-9 | 85-90 | b |
| Thermoscientific | Purification Kit | | | |
| Agro-Bio | EggsPure | 2-5 | 90 | c |
| GE Healthcare | HiTrap IgY | Information not | Information not | Information not |
| | Purification | available | available | available |
| Affiland | IgY Purification Kit | Information not | 98 | Information not |
| | | available | | available |

According to one embodiment of the method according to the invention, prior to immunization, one or more eggs of the bird which is to be immunized are collected. In such way, IgY purified from these eggs can be later used as control IgY for characterization purposes.

According to one embodiment of the method according to the invention, immunization takes place by means of injection of the protein or peptide into the breast tissue of the bird.

According to one embodiment of the method according to the invention, for immunization, a dosis of between ≥0.02 and ≤0.5 mg protein or peptide is injected to the bird.

According to one embodiment of the method according to the invention, the isolation or purification of the antibody comprises the steps of
a) optionally, skimming milk or colostrum by removing fat
b) removing casein e.g. by lowering pH to 4.6 by adding an acid, and
c) collecting the liquid phase and concentration or purification of the antibody fraction.

Such methods are e.g. described in Aich et al (2015), the content of which is incorporated herein by reference.

The polyclonal antibody molecules directed against the immunogenic protein can be isolated from the lactation product and further purified by well-known techniques, such as affinity chromatography using protein A or protein G, which process provides primarily the IgG fraction of comprised in the lactation product.

Subsequently, or alternatively, SARS CoV-2 spike protein, or a fragment thereof, is immobilized on a column or on beads, to act as a capturing agent to purify the respective antibody by immunoaffinity chromatography.

In one embodiment, said capturing agent comprises a sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41.

According to one embodiment of the method according to the invention, prior to immunization, one or more samples of milk, colostrum or whey of the mammal which is to be immunized are collected. In such way, antibodies purified therefrom can be later used as control for characterization purposes.

According to one embodiment of the method according to the invention, immunization takes place by means of injection of the protein or peptide by at least one of
intramuscular
subcutaneous
intramammary or
intravenous
infusion or injection.

In general, the immunization regimen occurs during the prepartum period of the female mammal, e.g., the cow. Cows, for example, will have their first calf early in their third year, marking the start of their first lactation. The cow will be re-bred about two to three months into lactation. Pregnancy is approximately 280 days. At about 2 months before expected calving date, or approximately 10 months into lactation, milk removal is halted and the cow is given what is called a dry period. The mammary gland undergoes a process of involution during the early dry period where most residual milk components are broken down and resorbed. The mammary gland begins a redevelopment phase several weeks prior to calving. Colostrum formation occurs in the days leading up to calving, coinciding with the early phase of. In the cow, lactogenesis begins shortly prior to calving and extends into the first few days postpartum.

Colostrum collected at the first milking of the cow after calving represents the accumulation of colostral products during the days leading up to parturition, including immunoglobulins which are at their highest concentration in the first milking.

Immunization protocols used to produce many immune milk products vary depending on the purpose, especially in the number and timing of immunizations.

When specifically collecting colostrum shortly after calving, multiple immunizations are administered during late pregnancy when the cow would be in the dry period. Mammary secretions then are collected either only at first milking, pooled from the first 4 to 6 milkings, pooled from the first 6 to 10 days after calving, or collected for longer periods into lactation.

Likewise, it is possible to initiate immunizations during the late dry period and then perform continued vaccinating throughout lactation, or only vaccinate during lactation.

According to one embodiment of the method according to the invention, for immunization, a dosis of between ≥0.05 and ≤2 mg protein or peptide is injected to the mammal

According to one embodiment of the method according to the invention, for immunization, an adjuvant is co-administered with the at least one protein or peptide.

Preferably, the adjuvant is at least selected from the group consisting of
Freund's complete or incomplete adjuvant
mineral gels (e.g., aluminum hydroxide),
surface-active substances (e.g., lysolecithin, pluronic polyols, polyanions, peptides, oil emulsions, dinitrophenol, etc.),
Bacille Calmette-Guerin, and/or
MPL-TDM adjuvant (monophosphoryl Lipid A, synthetic trehalose dicorynomycolate).

In chicken, the total volume of antigen/adjuvant should be about 1 ml with the adjuvant making up between half and two-thirds the volume.

According to one embodiment of the method according to the invention, immunization is repeated between one and 10 times. For prolonged antibody production, the bird or mammal can be immunized over a long period of time, with even more than 10 repeated immunizations

According to one embodiment of the method according to the invention, in the repeated immunization, the amount of the protein or peptide is reduced to between ≥70% and <10% of the initial dosis.

According to one embodiment of the method according to the invention, immunization is performed by in at least two steps, wherein,
a) in a first step, the bird or mammal is immunized by administration of at least one single chain protein or peptide which comprises two or more subsequences each comprising an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, and
b) in a second step performed after the first step the bird or mammal is immunized by administration of at least one monomer which comprises an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO 29.

As regards step a), such single chain protein or peptide which comprises two or more subsequences is preferably a concatemer as described herein elsewhere, according to any one of SEQ ID NO 30-35. These concatemers are shown in FIG. 8 as well as their subsequences (“building clocks”), which are disclosed as SEQ ID NO 36-41.

The concatemers have been chosen to comprise repetitions of epitopes binding of which by an antibody is deemed to have an inhibitory effect of virus propagation. By using several repeats of these epitopes a critical mass is achieved which renders the concatemer immunogenic, so that a significant immune response can be reached.

It is important to mention that the said concatemers can be linear and/or denaturated peptides proteins (for this reason, as shown in the sequence table herein, in some building blocks Cys has been substituted by Gly, to avoid the formation of disulfide bridges.

As regards step b), such monomer is preferably at least one selected from the group consisting of SEQ ID NO 1-5, SEQ ID NO 11-13, or SEQ ID NO 17-29. Such monomers are preferably fully folded proteins.

It is further important to understand that, beyond one of the general concepts disclosed herein, which is a method for isolating or producing antibodies in a lactation or ovulation product of a female vertebrate, such concept of immunizing, in a first step, a subject by administration of at least one single chain protein or peptide (preferably a concatemer) and, in a second step performed after the first step, immunizing the subject by administration of least one monomer can be used also for direct immunization of animals or humans, e.g., in order to generate immunity against the SARS CoV 2 virus, in order to create monoclonal antibodies by the hybridoma technique, or in order to vaccinate a human or animal. The advantages and preferred embodiment discussed herein elsewhere translate also to such applications.

According to one embodiment of the method according to the invention, at least one of the proteins or peptides used for immunization is produced by at least one of peptide synthesis or recombinant expression.

Peptides produced by synthesis are usually shorter than proteins and thereby only present a linear epitope in most cases. Larger peptides and proteins are classically produced by expression using recombinant technology.

Such larger peptides and proteins will form primary, secondary and also potentially tertiary folds, resembling that of the native protein and thereby beside linear epitopes also provide conformational epitopes. Though proteins are considered to be more immunogenic, especially large proteins have an unnecessary antigenic load that, that only contributes little to the protective immune response. Peptides often require particulate carriers for delivery and adjuvants to be immunogenic. Compared to proteins peptides on the other hand induces a highly targeted immune response giving the opportunity to direct it to areas where antibody binding interferes with the infectious process. Such a peptide based targeting may increase the chance to produce high titers of neutralizing antibodies. The same is true for proteins presenting domains of such function.

According to one embodiment of the method according to the invention, the recombinant expression is done in at least one of
a prokaryotic expression system, like e.g. E. coli or B. subtilis
a fungal or yeast based expression system, like S. cerevisiae
a protozoan expression system, like Tetrahymena thermophila
an baculovirus/insect expression system
a mammalian expression system, like CHO cells.

Prokaryotic expressions systems like E. coli and Bacillus for the production of recombinant protein, e.g. insulin in E. coli. Bacillus subtilis additionally shows the advantage of secreting recombinant proteins from cells into the media. Prokaryotic systems are relatively low in their but have the that eukaryotic post translational protein modifications as glycosylation is missing.

Fungal or yeast expression systems share the ease and low cost of production and also provide posttranslational modifications like glycosylation.

Protozoan expressions system like Tetrahymena thermophile represents a further eukaryotic expression system.

In a mammalian cell system, recombinant mammalian proteins can be produced that are no different from their natural counterparts. Disadvantages are the high costs of expression in such a system, which are due, among other things, to the effort of purity (production site, media) as well as the low space time yield of produced protein.

With regard to the SARS CoV-2 antigens to be expressed, the following should be noted:

An E. coli expression system is a very good system when the protein can be expressed in a soluble form. This system is the best alternative to quickly and cost-effectively obtain large amounts of antigen. The system is not able to glycosylate the proteins though.

A mammalian expression system could express the full length spikes protein of SARS CoV-2, as well as parts of it. These proteins would then be identical to those produced in humans, including glycosylation. If authentic glycosylation is an important issue for the immune response, this system would be the choice. A major disadvantage is the high cost of such a system, as well as the low yields. This can make such a project economically not feasible.

Further details of the production and purification process are disclosed, inter alia, in U.S. Pat. Nos. 8,173,783 and 9,873,732, the contents of which is incorporated herein by reference.

According another aspect of the invention, an antibody that binds specifically to a protein or peptide comprising a sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, wherein the protein or peptide is, comprises or is a fragment of the SARS CoV-2 spike protein.

According to another aspect of the invention, a combination of two or more antibodies that bind specifically to one or more proteins or peptides selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, wherein the proteins or peptides are, comprise or are a fragment of the SARS CoV-2 spike protein.

According another aspect of the invention, antibody
inhibits binding of SARS-Cov2 to the human ACE2 receptor, and/or
interferes with the priming of the spike protein by blocking proteolytic cleavage thereof at S1/S2 or S2′.

According to one embodiment of the invention, the antibody or antibodies has/have a neutralizing titer (NT₅₀) of ≤1×10⁵.

In preferred embodiments, the antibody/antibodies has/have a neutralizing titer (NT₅₀) of ≤5×10⁴; ≤5×10⁴; ≤1×10⁴; ≤5×10³; ≤1×10³; ≤5×10²; ≤1×10²; ≤5×10¹; or most preferred ≤1×10¹.

As used herein, the term “Neutralizing titer” defines the concentration of antibody to neutralize viral activity by 50% compared to the antibody free virus.

As used herein, the term “neutralize” refers to the ability of an antibody to bind to the virus, and reduce the biological activity, for example, virulence, of the infectious agent. The minimal requirement for neutralization is the ability for the antibody to bind to the infectious agent. In one embodiment, the antibody immunospecifically binds at least one specified epitope or antigenic determinant of the virus.

The neutralizing titer can be determined with a plaque reduction neutralization test. The solution of antibody to be tested is diluted and mixed with a viral suspension. This is incubated to allow the antibody to react with the virus. This is poured over a confluent monolayer of host cells. The surface of the cell layer is covered in a layer of agar or carboxymethyl cellulose to prevent the virus from spreading indiscriminately. The concentration of plaque forming units can be estimated by the number of plaques (regions of infected cells) formed after a few days. Depending on the virus, the plaque forming units are measured by microscopic observation, fluorescent antibodies or specific dyes that react with infected cells. The concentration of antibody to reduce the number of plaques by 50% compared to the antibody free virus gives the measure of how much antibody is present or how effective it is.

Currently it is considered to be the “gold standard” for detecting and measuring antibodies that can neutralise the viruses that cause many diseases.[2][3] It has a higher sensitivity than other tests like hemagglutination and many commercial Enzyme immunoassay without compromising specificity. Moreover, it is more specific than other serological methods for the diagnosis of some arbovirus.

However, the test is relatively cumbersome and time intensive (few days) relative to EIA kits that give quick results (usually several minutes to a few hours).

An issue with this assay that has recently been identified is that the neutralization ability of the antibodies is dependent on the virion maturation state and the cell-type used in the assay. [4] Therefore, if the wrong cell line is used for the assay it may seem that the antibodies have neutralization ability when they actually do not, or vice versa they may seem ineffective when they actually possess neutralization ability.

One assay for determining NT₅₀ is described in He et al. (2005), the content of which is incorporated by reference herein. Briefly, 293T cells are co transfected with a plasmid encoding SARS-CoV S protein and a plasmid encoding Env-defective, luciferase-expressing HIV-1 genome (pNL4-3.luc.RE) using FuGENE6 transfection reagents (RocheApplied Science). Supernatant is harvested 72 h post-transfection and used for single-cycle infection of ACE2/293T cells. The virus containing supernatant is preincubated with a dilution series comprising the antibody according to the invention at 37° C. for 1 h before adding to the cells. Fresh medium is added 24 h later, followed by lysing cells using cell lysis buffer (Promega). After addition of luciferase substrate (Promega), relative luciferase activity is determined in Ultra 384 luminometer (Tecan). SARS Virus neutralization is calculated and expressed as 50% neutralizing antibody titer, NT50 (Chou, 2006, the content of which is incorporated by reference herein).

According to another assay for determining NT₅₀, a dilution series comprising the antibody according to the invention is mixed with equal volumes of SARS-CoV-2 and incubated at 37 1C for 1 h. Vero E6 cells are then infected with 100 mL of the virus-antibody mixtures in 96-well plates. After 6 days of incubation, the neutralization titer is determined as the endpoint dilution of the antibody at which there was 50% inhibition of the SARS-CoV-induced cytopathic effect. The assay is described in described in Yusuhi et al (2014), the content of which is incorporated herein by reference.

According to one embodiment of the invention, the antibody or antibodies compete/competes with ACE2 for binding to the SARS-Cov2 spike protein.

According to one embodiment of the invention, the antibody or antibodies is a polyclonal antibody/are polyclonal antibodies.

In one embodiment, when purified from eggs, the antibody is a polyclonal IgY antibody. In another embodiment, when purified from milk, whey or colostrum the antibody is an IgG, IgG or IgM.

According to one embodiment of the invention, the antibody or antibodies is/are produced by the method according to the above description.

According to another aspect of the invention, a product comprising at least one antibody according to the above description is provided, the product being provided as at least one selected from the group consisting of
Lozenge
Chewing gum
Tablet
Cream or gel
Gargling solution or mouthwash
liquid solution for topic application
aerosol for intraoral, intratracheal or intranasal administration, and/or
aqueous solution for intravenous, subcutaneous or intramuscular administration

According to one embodiment of the invention, said product is embedded or provided in at least one item selected from the group consisting of:
Breathing mask
Filter for fluid media, including gases and liquids, and/or
Disinfectant gel, liquid, aerosol or spray.

According to one aspect of the invention, the antibody or antibodies according to the above description are provided for (the manufacture of a medicament) for use in the treatment of a patient that suffers from or diagnosed for SARS Cov-2 infection, or for the prevention of such condition.

According to one aspect of the invention, the product according to the above description is provided for use in the treatment of a patient that suffers from or diagnosed for SARS Cov-2 infection, or for the prevention of such condition.

According to one aspect of the invention, in the antibody or antibodies or product according to the above descriptions, the antibody or antibodies is co-administered with, or the product further comprises, at least one of
a protease inhibitor, preferably an inhibitor of TMPRSS2 or an inhibitor of the CoV-2 C30 endopeptidase (also called Mpro or 3CLpro)
a nucleoside or nucleotide analogue, preferably which is accepted by viral RNA-dependent RNA polymerase
an IL-6 antagonist,
a soluble angiotensin-converting enzyme 2,
an anti rheumatic, and/or
a peptide that comprises a sequence that is homologous to the S1/S2 sequence of the SarsCov2 spike protein,

wherein co-administration can be carried out concomitantly or consecutively.

Nucleoside analogues and Nucleotide analogs correspond to their nucleotide or nucleotide counterparts, and are accepted by a given DNA- or RNA polymerase or transcriptase, but are chemically modified in such way that the polymerase process or the transcriptase process is inhibited or hampered.

One preferred nucleoside analogue is 2-Ethylbutyl-(2S)-2-{[(S)-{[(2R,3S,4R,5R)-5-(4-amino-pyrrolo[2,1-f][1,2,4]triazin-7-yl)-5-cyan-3,4-dihydroxytetrahydro-2-furanyl]methoxy}(phenoxy)phosphoryl]amino}propanoa (INN: remdesivir).

One other preferred nucleoside analogue is 1-[(2R,3R,4R,5R)-3,4-Dihydroxy-5-hydroxymethyl-oxolan-2-yl]-1,2,4-triazol-3-carboxamid (INN: ribavirin)

Another preferred nucleoside analogue is 6-Fluor-3-hydroxy-2-pyrazincarboxamid (INN: Favipiravir).

One preferred inhibitor of TMPRSS2 is (4-{2-[2-(Dimethylamino)-2-oxoethoxy]-2-oxoethyl}phenyl)(4-carbamimidamidobenzoat) (INN: Camostat)

One preferred inhibitor of CoV-2 C30 endopeptidase are the α-ketoamides like α-ketoamide 13b, as described in Zhang et al. (2020), the content of which is incorporated herein by reference. Another preferred inhibitor of CoV-2 C30 endopeptidase is lopinavir.

IL-6 antagonists interfere within the IL-6 signalling pathway, for example by binding to either IL6 or its receptor, IL-6R. One preferred IL-6 antagonist is the antibody Tocilizumab, also known as atlizumab, which is a humanized monoclonal antibody against the interleukin-6 receptor (IL-6R). Another preferred IL-6 antagonist is Sarilumab, which is a human monoclonal antibody against the interleukin-6 receptor

Soluble angiotensin-converting enzyme 2 has been described as a potential approach for coronavirus infection therapy in Batlle et al. (2020), the content of which is incorporated herein by reference.

Suitable anirheumatics include, but are not limited, to chloroquine/hydroxychloroquine, TNF antagonosts like Adalimumab, Etanercept, Infliximab Certolizumab Pegol and Golimumab, or Janus Kinase inhibitors like Baricitinib.

A peptide that comprises a sequence that is homologous to the S1/S2 sequence of the SarsCov2 spike protein can comprise an amino acid sequence according to SEQ ID NO: 6 or 7, or a fragment thereof. In such way, the host's TMPRSS2 can be competitively inhibited so as to avoid cleavage of the SARS CoV-2 spike protein. In one embodiment, such peptide can comprise one or more D amino acid residues to avoid cleavage thereof and hence increase the competitive inhibition.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus.

### Example 1: SARS-CoV2 Spike Protein Binding

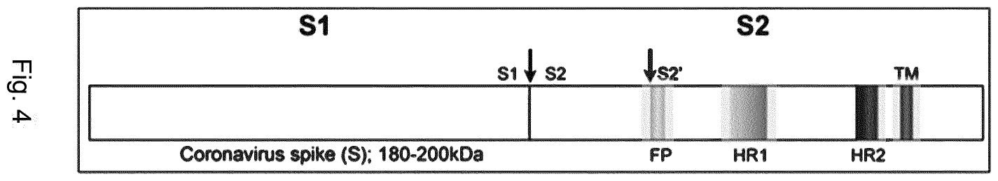

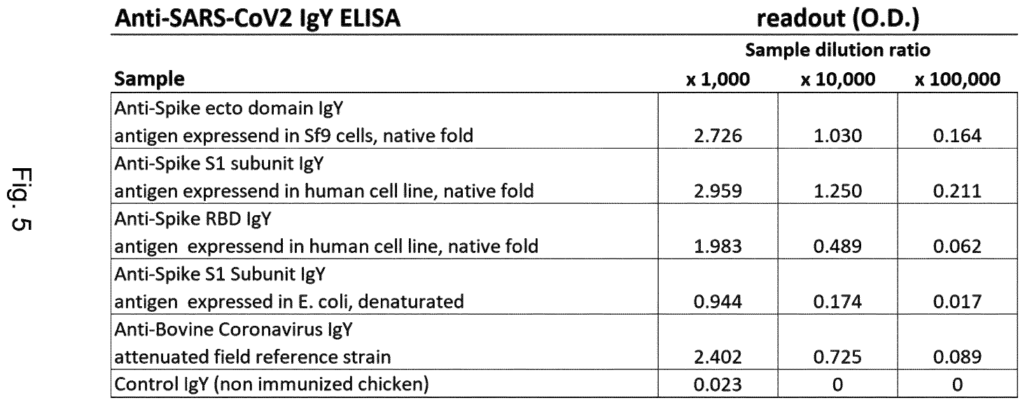

A 96-well plate has been precoated with SARS-CoV2 spike protein. Controls or test samples at different dilutions are added to the wells and incubated. Following washing, a horseradish peroxidase (HRP) labelled anti-Chicken IgG conjugate is added to the wells, which binds to the immobilized anti SARS-CoV2 spike protein specific antibodies. Binding is than analyzed using HRP quantification systems chromogenic or chemiluminescent according to standard protocols. Results are shown in FIG. 5.

### Example 2: CPE Neutralization Assay

The CPE (Cytopathic Effect) neutralization assay is a widely-employed assay format to screen for antiviral agents and neutralizing immunoglobulins. In this assay, host cell death of susceptible cells is a consequence of the viral infection and cell viability is a surrogate readout. Several assays can be used to test the viability of cells. In this case non-viable cells were removed from the confluet monolayer of host cells by washing and viable cells stained with methylene blue. Consequently, positive immunoglobulin preparations are those that protect the host cells from viral CPE.

A normalized number of infectious SARS-CoV-2 virions is incubated with serial dilutions of the chicken IgY antibody preparations. After a one-hour neutralization period of the virion/IgY antibody mixtures, it was inoculated to a 96well plate containing a confluent cell monolayer of host cells. Host cells used in this case were VERO E6 cells. Plates were incubated for 72 hours at 37° C. in a humidified atmosphere with 5% CO2. After incubation non-viable cells were removed by washing with an appropriate buffer, whereas not infected, intact cells were retained. Retained, viable cells were fixed in the well with methanol and stained with Methylene blue.

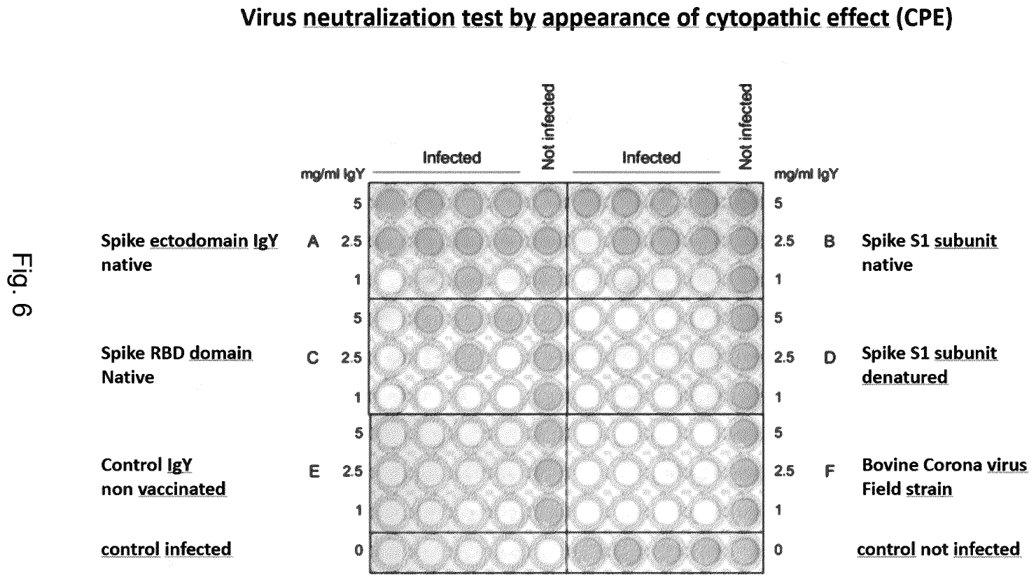

The higher the antibody dilution that can completely inhibit CPE formation, the more potent the antibody is considered to be. Results are shown in FIG. 6.

### Example 3: Fluorescent Plaque Reduction Assay

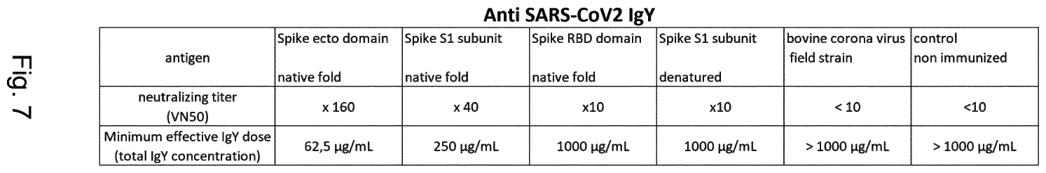

In this plaque reduction neutralization test PRNT, a standard number of infectious SARS-CoV-2 units was incubated with serial dilutions of the chicken IgY antibody preparations. After a one-hour neutralization period of the virus/IgY antibody mixtures, 100 μL of the mixture was added to a confluent monolayer of susceptible Vero cells for 16 hours (±2 hours). After this incubation period, the cells were fixed and virus-infected cells were immunostained with a SARS-CoV-2 specific polyclonal antibody, followed by a secondary goat anti-rabbit IgG Alexa Fluor-555 conjugate. Images of all wells were analyzed with a Cell Imaging Multi-Mode Reader (BioTek). The 50% neutralization titers (VN50) were calculated according to the method described by Zielinska, et al. Virol J. 2005. Results are shown in FIG. 7.

### Example 4: Purification of Spike Proteins from Komagataella Phaffii

A Komagataella phaffii Δoch1 strain with an integrated sequence encoding for the C-terminally His-tagged full-length protein (AA 15-1213), S1 domain (AA 15-685) and the receptor binding domain (AA 331-524) of the Sars-Cov2 spike protein were cultivated for 6 days at 30° C. with 150 rpm. The cells were harvested with 5000 g for 30 min at 4° C. and the resulting supernatant was then further microfiltrated (0.2 μm) and ultrafiltrated (10 kDa cutoff). The ultraconcentrated sample was then purified using Ni-NTA affinity chromatography to apparent homogeneity (>95% purity confirmed by SDS-PAGE). The elution fractions were pooled, concentrated using a Vivaspin Turbo 15 ultrafiltration centrifugal concentrator (10 kDa cutoff) and rebuffered to 100 mM Tris pH 7.8, 1 mM CaCl₂), 2 mM TCEP.

### Example 5: Purification of Spike Proteins from Sf9 Insect Cells

Sf9 insect cells were infected with a recombinant Baculovirus encoding for the C-terminally His-tagged full-length protein (AA 15-1213), 51 domain (AA 15-685) and the receptor binding domain (AA 319-541 and AA 319-591) of the Sars-Cov2 spike protein. The full-length protein contains at the C-terminus a fold on motif for trimerization, followed by a 6× His-tag.

The AA K986 and V987 in the motif DKVE were changed to Prolin and the AA of the furin cleavage side 682-685 were changed to AGAG. All spiked protein constructs were secreted into the medium with the help of the gp67 secretion signal.

After infection the cells were cultivated for 4 days at 27° C. with 90 rpm. The cells were harvested with 5000 g for 30 min at 4° C. and the resulting supernatant was then further microfiltrated (0.2 μm) and ultrafiltrated (10 kDa cutoff). The ultraconcentrated sample was then purified using Ni-NTA affinity chromatography to apparent homogeneity (>95% purity confirmed by SDS-PAGE). The elution fractions were pooled, concentrated using a Vivaspin Turbo 15 ultrafiltration centrifugal concentrator (10 kDa cutoff) and rebuffered to 100 mM Tris pH 7.8, 1 mM CaCl₂), 2 mM TCEP.

### Example 6: Purification of Concatemers

Artificial concatemers containing the repetitive amino acid sequences from six distinct parts (see sequence) of the Sars-Cov2 spike protein were recombinantly expressed as inclusion bodies (IBs) in Escherichia coli. The E. coli BL21 (DE3) strain containing a pET28a plasmid encoding for one concatemer was cultivated at 37° C. 150 rpm up to an OD600 of 0.6 and gene expression was then induced with 0.25 mM IPTG. The cells were further grown at 30° C. 150 rpm for 18 h. The cells were harvested with 5000 g for 30 min at 4° C., and then lysed using Bugbuster Mastermix (Merck, Darmstadt) for 1 h followed by sonification for 5 min with max. power. The lysed cells were centrifuged with 20000 g for 30 min at 4° C. and the resulting supernatant was discarded. The IBs were washed 2-3×using 100 mM Tris pH 7.8, 1 mM CaCl₂), 2 mM TCEP, 2 M Urea, 5% Triton X100 to remove all cell membranes, lipids and membrane-associated proteins. A centrifugation step for 20000 g for 30 min 4° C. was done after each washing step. The IBs were then washed 1× using 100 mM Tris pH 7.8, 1 mM CaCl₂), 2 mM TCEP to remove urea and Triton X100 followed by a centrifugation step with 20000 g for 30 min at 4° C. The purified IBs were resolubilized using 100 mM Tris pH 7.8, 1 mM CaCl₂), 2 mM TCEP, 8 M guanidinium chloride. The solubilized IBs were then centrifuged with 20000 g for 30 min at 4° C. The resulting supernatant was then filtered using 0.2 μm filter and further purified using a denaturizing Ni-NTA affinity chromatography. The elution fractions were pooled, concentrated using a Vivaspin Turbo 15 ultrafiltration centrifugal concentrators (10 kDa cutoff) and rebuffered to 100 mM Tris pH 7.8, 1 mM CaCl₂), 2 mM TCEP, 4 M guanidine chloride.

The concatemers are shown in FIG. 8

### Example 7: SARS-CoV-2 Viral Neutralization Assay

Dissolving the Lyophilized Test Items

The lyophilized IgY antibody preparations will be dissolved in pre-warmed DMEM+2% FBS+Pen-Strep-G by shaking at 37° C. for 15-30 minutes. If not all of the lyophilized preparation is dissolved, then the IgY antibody-DMEM solutions will be centrifuged at 3000 rpm for 10 minutes at RT to remove any undissolved particles. The IgY antibody-DMEM solutions will be passed through a 0.45-micron filter, before testing these IgY solutions in the viral neutralization tests.

Determining the Starting Concentration of Each IgY Preparations

As the chicken IgY antibody preparations at high concentrations may be toxic to the Vero cells, a cell toxicity assay will be performed. Vero cells will be incubated with serial dilutions of each of the chicken IgY antibody preparations for 3 days. The first concentration of these serial dilutions will be 5 mg/ml. After 3 days, the cell toxicity will be monitored. The highest concentration of each IgY antibody preparation that does not cause cell toxicity will be used as the starting concentration in the viral neutralization tests.

Description of the Test System

The ability of the chicken IgY antibody preparations to suppress the formation of plaques (infection) will be determined via a plaque reduction (PRNT) and a CPE-based viral neutralization assay.

CPE-Based Viral Neutralization Assay

In this CPE-based viral neutralization assay, a standard number of infectious SARS-CoV-2 units (Wuhan or B.1.1.7) is incubated with serial dilutions of the chicken IgY antibody preparations. After a one-hour neutralization period of the virus/IgY antibody mixtures, 100 μL of the mixture is added to a confluent monolayer of susceptible Vero cells for 3 days. After this incubation period, the ability of the chicken IgY antibody preparations to inhibit the development of CPE by 100% will be determined by screening the cells for the presence of CPE. The higher the antibody dilution that can completely inhibit CPE formation, the more potent the antibody is considered to be.

PRNT

In this PRNT, a standard number of infectious SARS-CoV-2 units (Wuhan or B.1.1.7) is incubated with serial dilutions of the chicken IgY antibody preparations. After a one-hour neutralization period of the virus/IgY antibody mixtures, 100 μL of the mixture is added to a confluent monolayer of susceptible Vero cells for 16 hours (±2 hours). After this incubation period, the cells are fixed and virus-infected cells are immunostained with a SARS-CoV-2 specific polyclonal antibody, followed by a secondary goat anti-rabbit IgG Alexa Fluor-555 conjugate. Images of all wells will be analyzed using the Cytation™ 1 Cell Imaging Multi-Mode Reader (BioTek) equipped with software to quantitate the SARS-CoV-2-positive cells. The 50% and 80% neutralization titers are calculated according to the method described by Zielinska, E et al. Virol J. 2005.

### REFERENCES

The disclosures of these documents are herein incorporated by reference in their entireties.
Kovacs-Nolan, J. and Y. Mine, 2004. Avian and Poultry Biology Reviews 15 (1), 2004, 25-46.
Hoffmann et al., 2020. Cell 181, pp 181-280 Akita, E M and S. Nakai, 1993. J. Immunol. Methods 160: 207-14.
Tan S H, Mohamedali A, Kapur A, Lukjanenko L, Baker M S, 2012. J Immunol Methods. J29; 380(1-2):73-6.
Stålberg and Larsson, 2001. Upsala J Med Sci 106: 99-110
Petr Hodek, Pavel Trefil, Jiri Simunek, Jiri Hudecek, Marie Stiborova, 2013. Int. J. Electrochem. Sci., 8, 113-124.
Aich R, Batabyal S Joardar S N, 2015. Vet World. May; 8(5):621-4.
Batlle, Daniel & Wysocki, Jan & Satchell, Karla. (2020) Clinical Science. 134. 543-545.
Yasui, Fumihiko & Kohara, Michinori & Kitabatake, Masahiro & Nishiwaki, Tetsu & Fujii, Hideki & Tateno, Chise & Yoneda, Misako & Morita, Kouichi & Matsushima, Kouji & Koyasu, Shigeo & Kai, Chieko, 2014. Virology. 451/155. 157-168.
Linlin Zhang, Daizong Lin, Xinyuanyuan Sun, Ute Curth, Christian Drosten, Lucie Sauerhering, Stephan Becker, Katharina Rox, Rolf Hilgenfeld, 2020. Science 24, 409-412
Ting-Chao Chou Pharmacological Reviews September 2006, 58 (3) 621-681
Yuxian He, Hong Lu, Pamela Siddiqui, Yusen Zhou and Shibo Jiang: J Immunol Apr. 15, 2005, 174 (8) 4908-4915
Zielinska, E et al. Virol J. 2005.

Sequences

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones. Nucleotide sequences are only disclosed in the electronic sequence listing.

Note that SEQ ID NOs 1-10 correspond to the wildtype spike protein, or fragments or epitopes thereof. Note that SEQ ID NOs 11-15 comprise one or two substitutions relative to SEQ ID NOs 1, 2, 5, 6 or 7.

In SEQ ID NOs: 11, 12, 13 and 15, the Arginine residue (R) in the peptide motif “ARS” as in the corresponding wildtype protein or peptide has been replaced by an Alanine residue (A), to arrive at the peptide motif “AAS”.

The same applies to SEQ ID NO: 11, 12, and 14, where the Arginine residue (R) in the peptide motif “KRS” as in the corresponding wildtype protein or peptide has been replaced by an Alanine residue (A), to arrive at the peptide motif “KAS”.

The peptide motifs AR and KR, as in the corresponding wildtype protein or peptide represent a cleavage site for proteases, like e.g. TMPRSS2 (transmembrane serine protease type 2) and other related proteases. To stabilize the position against proteolytic cleavage, the inventors have mutated the respective sites in the proteins and peptides used for immunization. For explanation of the terms “S1” and “S2” see elsewhere in the text.

It should be noted that SEQ ID NO: 11 and 12 comprise two substitutions (ARS→AAS and KRS→KAS). However, applicant deems that variants of SEQ ID NO: 11 and 12 which have only one of the two substitutions are likewise useful and should hence deemed to be disclosed, and encompassed by the scope of the present invention.

It should also be noted that some sequences comprise secretion signal peptides which are of always necessary for immunogenicity. These sequences shall be deemed disclosed with and without sch secretion signal peptides

It should be also be noted that some SEQ ID NOs comprise, in the motif DKVE, a substitution of K and V to P each. However, applicant deems that variants which have DKVE shall also be deemed disclosed as having DPPE instead, and vice versa.

| | | | | AAs |
|---|---|---|---|---|
| SEQ | | | | within |
| ID | | domain/ | Length | SEQ ID |
| NO | AA sequence | epitope | (AA) | NO: 1 |
| 1 | MFVFLVLLPLVSSQCVNLTTRTQLPPAYTNSFTRGVYYPDKVFR | Full length SARS- | 1273 | |
| | SSVLHSTQDLFLPFFSNVTWFHAIHVSGTNGTKRFDNPVLPFN | CoV-2 Spike protein | | |
| | DGVYFASTEKSNIIRGWIFGTTLDSKTQSLLIVNNATNWIKVCE | 1-12 secretion | | |
| | FQFCNDPFLGVYYHKNNKSWMESEFRVYSSANNCTFEYVSQP | signal | | |
| | FLMDLEGKQGNFKNLREFVFKNIDGYFKIYSKHTPINLVRDLPQ | | | |
| | GFSALEPLVDLPIGINITRFQTLLALHRSYLTPGDSSSGWTAGAA | | | |
| | AYYVGYLQPRTFLLKYNENGTITDAVDCALDPLSETKCTLKSFTV | | | |
| | EKGIYQTSNFRVQPTESIVRFPNITNLCPFGEVFNATRFASVYA | | | |
| | WNRKRISNCVADYSVLYNSASFSTFKCYGVSPTKLNDLCFTNVY | | | |
| | ADSFVIRGDEVRQIAPGQTGKIADYNYKLPDDFTGCVIAWNSN | | | |
| | NLDSKVGGNYNYLYRLFRKSNLKPFERDISTEIYQAGSTPCNGVE | | | |
| | GFNCYFPLQSYGFQPTNGVGYQPYRVVVLSFELLHAPATVCGP | | | |
| | KKSTNLVKNKCVNFNFNGLTGTGVLTESNKKFLPFQQFGRDIA | | | |
| | DTTDAVRDPQTLEILDITPCSFGGVSVITPGTNTSNQVAVLYQD | | | |
| | VNCTEVPVAIHADQLTPTWRVYSTGSNVFQTRAGCLIGAEHVN | | | |
| | NSYECDIPIGAGICASYQTQTNSPRRARSVASQSIIAYTMSLGAE | | | |
| | NSVAYSNNSIAIPTNFTISVTTEILPVSMTKTSVDCTMYICGDSTE | | | |
| | CSNLLLQYGSFCTQLNRALTGIAVEQDKNTQEVFAQVKQIYKTP | | | |
| | PIKDFGGFNFSQILPDPSKPSKRSFIEDLLFNKVTLADAGFIKQYG | | | |
| | DCLGDIAARDLICAQKFNGLTVLPPLLTDEMIAQYTSALLAGTIT | | | |
| | SGWTFGAGAALQIPFAMQMAYRFNGIGVTQNVLYENQKLIA | | | |
| | NQFNSAIGKIQDSLSSTASALGKLQDVVNQNAQALNTLVKQLS | | | |
| | SNFGAISSVLNDILSRLDKVEAEVQIDRLITGRLQSLQTYVTQQLI | | | |
| | RAAEIRASANLAATKMSECVLGQSKRVDFCGKGYHLMSFPQSA | | | |
| | PHGVVFLHVTYVPAQEKNFTTAPAICHDGKAHFPREGVFVSNG | | | |
| | THWFVTQRNFYEPQIITTDNTFVSGNCDVVIGIVNNTVYDPLQ | | | |
| | PELDSFKEELDKYFKNHTSPDVDLGDISGINASVVNIQKEIDRLN | | | |
| | EVAKNLNESLIDLQELGKYEQYIKWPWYIWLGFIAGLIAIVMVTI | | | |
| | MLCCMTSCCSCLKGCCSCGSCCKFDEDDSEPVLKGVKLHYT | | | |
| 2 | VNLTTRTQLPPAYTNSFTRGVYYPDKVFRSSVLHSTQDLFLPFFS | Full length S1/S2 | 1198 | 16 to |
| | NVTWFHAIHVSGTNGTKRFDNPVLPFNDGVYFASTEKSNIIRG | spike ectodomain | | 1213 |
| | WIFGTTLDSKTQSLLIVNNATNVVIKVCEFQFCNDPFLGVYYHK | | | |
| | NNKSWMESEFRVYSSANNCTFEYVSQPFLMDLEGKQGNFKNLRE | | | |
| | FVFKNIDGYFKIYSKHTPINLVRDLPQGFSALEPLVDLPIGINIT | | | |
| | RFQTLLALHRSYLTPGDSSSGWTAGAAAYYVGYLQPRTFLLKYN | | | |
| | ENGTITDAVDCALDPLSETKCTLKSFTVEKGIYQTSNFRVQPTESI | | | |
| | VRFPNITNLCPFGEVFNATRFASVYAWNRKRISNCVADYSVLYN | | | |
| | SASFSTFKCYGVSPTKLNDLCFTNVYADSFVIRGDEVRQIAPGQT | | | |
| | GKIADYNYKLPDDFTGCVIAWNSNNLDSKVGGNYNYLYRLFRK | | | |
| | SNLKPFERDISTEIYQAGSTPCNGVEGFNCYFPLQSYGFQPTNG | | | |
| | VGYQPYRVVVLSFELLHAPATVCGPKKSTNLVKNKCVNFNFNG | | | |
| | LTGTGVLTESNKKFLPFQQFGRDIADTTDAVRDPQTLEILDITPC | | | |
| | SFGGVSVITPGTNTSNQVAVLYQDVNCTEVPVAIHADQLTPTW | | | |
| | RVYSTGSNVFQTRAGCLIGAEHVNNSYECDIPIGAGICASYQTQ | | | |
| | TNSPRRARSVASQSIIAYTMSLGAENSVAYSNNSIAIPTNFTISVT | | | |
| | TEILPVSMTKTSVDCTMYICGDSTECSNLLLQYGSFCTQLNRALT | | | |
| | GIAVEQDKNTQEVFAQVKQIYKTPPIKDFGGFNFSQILPDPSKP | | | |
| | SKRSFIEDLLFNKVTLADAGFIKQYGDCLGDIAARDLICAQKFNG | | | |
| | LTVLPPLLTDEMIAQYTSALLAGTITSGWTFGAGAALQIPFAMQ | | | |
| | MAYRFNGIGVTQNVLYENQKLIANQFNSAIGKIQDSLSSTASAL | | | |
| | GKLQDVVNQNAQALNTLVKQLSSNFGAISSVLNDILSRLDKVE | | | |
| | AEVQIDRLITGRLQSLQTYVTQQLIRAAEIRASANLAATKMSECV | | | |
| | LGQSKRVDFCGKGYHLMSFPQSAPHGVVFLHVTYVPAQEKNF | | | |
| | TTAPAICHDGKAHFPREGVFVSNGTHWFVTQRNFYEPQIITTD | | | |
| | NTFVSGNCDVVIGIVNNTVYDPLQPELDSFKEELDKYFKNHTSP | | | |
| | DVDLGDISGINASVVNIQKEIDRLNEVAKNLNESLIDLQELGKYE | | | |
| | QYIKWP | | | |
| 3 | RVQPTESIVRFPNITNLCPFGEVFNATRFASVYAWNRKRISNCV | Binding Domain | 273 | 319 to |
| | ADYSVLYNSASFSTFKCYGVSPTKLNDLCFTNVYADSFVIRGDEV | fragment | | 591 |
| | RQIAPGQTGKIADYNYKLPDDFTGCVIAWNSNNLDSKVGGNY | | | |
| | NYLYRLFRKSNLKPFERDISTEIYQAGSTPCNGVEGFNCYFPLQS | | | |
| | YGFQPTNGVGYQPYRVVVLSFELLHAPATVCGPKKSTNLVKNK | | | |
| | CVNFNFNGLTGTGVLTESNKKFLPFQQFGRDIADTTDAVRDPQ | | | |
| | TLEILDITPCS | | | |
| 4 | PNITNLCPFGEVFNATRFASVYAWNRKRISNCVADYSVLYNSAS | Short Binding | 201 | 330 to |
| | FSTFKCYGVSPTKLNDLCFTNVYADSFVIRGDEVRQIAPGQTGKI | Domain fragment | | 530 |
| | ADYNYKLPDDFTGCVIAWNSNNLDSKVGGNYNYLYRLFRKSNL | | | |
| | KPFERDISTEIYQAGSTPCNGVEGFNCYFPLQSYGFQPTNGVGY | | | |
| | QPYRVVVLSFELLHAPATVCGPKKS | | | |
| 5 | YECDIPIGAGICASYQTQTNSPRRARSVASQSIIAYTMSLGAENS | Protease cleavage | 127 | 660 to |
| | VAYSNNSIAIPTNFTISVTTEILPVSMTKTSVDCTMYICGDSTECS | domain S1/S2 | | 786 |
| | NLLLQYGSFCTQLNRALTGIAVEQDKNTQEVFAQVK | | | |
| 6 | LPDPSKPSKRSFIEDLL | S2′proteolytic site | 17 | 806 to |
| | | epitope | | 822 |
| 7 | QTNSPRRARSVASQSIIA | S1/S2 proteolytic | 18 | 677 to |
| | | site epitope | | 694 |
| 8 | SNNLDSKVGGN | Binding motif loop | 11 | 438 to |
| | | 1a epitope | | 448 |
| 9 | QPTNGVGYQP | Binding motif loop | 10 | 498 to |
| | | 1b epitope | | 507 |
| 10 | QAGSTPCNGVE | Binding motif loop | 11 | 474 to |
| | | 2 epitope | | 484 |
| 11 | MFVFLVLLPLVSSQCVNLTTRTQLPPAYTNSFTRGVYYPDKVFR | Full length SARS- | 1273 | |
| | SSVLHSTQDLFLPFFSNVTWFHAIHVSGTNGTKRFDNPVLPFN | CoV-2 Spike protein | | |
| | DGVYFASTEKSNIIRGWIFGTTLDSKTQSLLIVNNATNVVIKVCE | with R in the | | |
| | FQFCNDPFLGVYYHKNNKSWMESEFRVYSSANNCTFEYVSQP | wildtype peptide | | |
| | FLMDLEGKQGNFKNLREFVFKNIDGYFKIYSKHTPINLVRDLPQ | motif ARS or KRS | | |
| | GFSALEPLVDLPIGINITRFQTLLALHRSYLTPGDSSSGWTAGAA | replaced by A | | |
| | AYYVGYLQPRTFLLKYNENGTITDAVDCALDPLSETKCTLKSFTV | 1-12secretion | | |
| | EKGIYQTSNFRVQPTESIVRFPNITNLCPFGEVFNATRFASVYA | signal | | |
| | WNRKRISNCVADYSVLYNSASFSTFKCYGVSPTKLNDLCFTNVY | | | |
| | ADSFVIRGDEVRQIAPGQTGKIADYNYKLPDDFTGCVIAWNSN | | | |
| | NLDSKVGGNYNYLYRLFRKSNLKPFERDISTEIYQAGSTPCNGVE | | | |
| | GFNCYFPLQSYGFQPTNGVGYQPYRVVVLSFELLHAPATVCGP | | | |
| | KKSTNLVKNKCVNFNFNGLTGTGVLTESNKKFLPFQQFGRDIA | | | |
| | DTTDAVRDPQTLEILDITPCSFGGVSVITPGTNTSNQVAVLYQD | | | |
| | VNCTEVPVAIHADQLTPTWRVYSTGSNVFQTRAGCLIGAEHVN | | | |
| | NSYECDIPIGAGICASYQTQTNSPRRAASVASQSIIAYTMSLGAE | | | |
| | NSVAYSNNSIAIPTNFTISVTTEILPVSMTKTSVDCTMYICGDSTE | | | |
| | CSNLLLQYGSFCTQLNRALTGIAVEQDKNTQEVFAQVKQIYKTP | | | |
| | PIKDFGGFNFSQILPDPSKPSKASFIEDLLFNKVTLADAGFIKQYG | | | |
| | DCLGDIAARDLICAQKFNGLTVLPPLLTDEMIAQYTSALLAGTIT | | | |
| | SGWTFGAGAALQIPFAMQMAYRFNGIGVTQNVLYENQKLIA | | | |
| | NQFNSAIGKIQDSLSSTASALGKLQDVVNQNAQALNTLVKQLS | | | |
| | SNFGAISSVLNDILSRLDKVEAEVQIDRLITGRLQSLQTYVTQQLI | | | |
| | RAAEIRASANLAATKMSECVLGQSKRVDFCGKGYHLMSFPQSA | | | |
| | PHGVVFLHVTYVPAQEKNFTTAPAICHDGKAHFPREGVFVSNG | | | |
| | THWFVTQRNFYEPQIITTDNTFVSGNCDVVIGIVNNTVYDPLQ | | | |
| | PELDSFKEELDKYFKNHTSPDVDLGDISGINASVVNIQKEIDRLN | | | |
| | EVAKNLNESLIDLQELGKYEQYIKWPWYIWLGFIAGLIAIVMVTI | | | |
| | MLCCMTSCCSCLKGCCSCGSCCKFDEDDSEPVLKGVKLHYT | | | |
| 12 | VNLTTRTQLPPAYTNSFTRGVYYPDKVFRSSVLHSTQDLFLPFFS | Full length S1/S2 | 1198 | 16 to |
| | NVTWFHAIHVSGTNGTKRFDNPVLPFNDGVYFASTEKSNIIRG | spike ectodomain | | 1213 |
| | WIFGTTLDSKTQSLLIVNNATNVVIKVCEFQFCNDPFLGVYYHK | with R in the | | |
| | NNKSWMESEFRVYSSANNCTFEYVSQPFLMDLEGKQGNFKNLRE | wildtype peptide | | |
| | FVFKNIDGYFKIYSKHTPINLVRDLPQGFSALEPLVDLPIGINIT | motif ARS or KRS | | |
| | RFQTLLALHRSYLTPGDSSSGWTAGAAAYYVGYLQPRTFLLKYN | replaced by A | | |
| | ENGTITDAVDCALDPLSETKCTLKSFTVEKGIYQTSNFRVQPTESI | | | |
| | VRFPNITNLCPFGEVFNATRFASVYAWNRKRISNCVADYSVLYN | | | |
| | SASFSTFKCYGVSPTKLNDLCFTNVYADSFVIRGDEVRQIAPGQT | | | |
| | GKIADYNYKLPDDFTGCVIAWNSNNLDSKVGGNYNYLYRLFRK | | | |
| | SNLKPFERDISTEIYQAGSTPCNGVEGFNCYFPLQSYGFQPTNG | | | |
| | VGYQPYRVVVLSFELLHAPATVCGPKKSTNLVKNKCVNFNFNG | | | |
| | LTGTGVLTESNKKFLPFQQFGRDIADTTDAVRDPQTLEILDITPC | | | |
| | SFGGVSVITPGTNTSNQVAVLYQDVNCTEVPVAIHADQLTPTW | | | |
| | RVYSTGSNVFQTRAGCLIGAEHVNNSYECDIPIGAGICASYQTQ | | | |
| | TNSPRRAASVASQSIIAYTMSLGAENSVAYSNNSIAIPTNFTISVT | | | |
| | TEILPVSMTKTSVDCTMYICGDSTECSNLLLQYGSFCTQLNRALT | | | |
| | GIAVEQDKNTQEVFAQVKQIYKTPPIKDFGGFNFSQILPDPSKP | | | |
| | SKASFIEDLLFNKVTLADAGFIKQYGDCLGDIAARDLICAQKFNG | | | |
| | LTVLPPLLTDEMIAQYTSALLAGTITSGWTFGAGAALQIPFAMQ | | | |
| | MAYRFNGIGVTQNVLYENQKLIANQFNSAIGKIQDSLSSTASAL | | | |
| | GKLQDVVNQNAQALNTLVKQLSSNFGAISSVLNDILSRLDKVE | | | |
| | AEVQIDRLITGRLQSLQTYVTQQLIRAAEIRASANLAATKMSECV | | | |
| | LGQSKRVDFCGKGYHLMSFPQSAPHGVVFLHVTYVPAQEKNF | | | |
| | TTAPAICHDGKAHFPREGVFVSNGTHWFVTQRNFYEPQIITTD | | | |
| | NTFVSGNCDVVIGIVNNTVYDPLQPELDSFKEELDKYFKNHTSP | | | |
| | DVDLGDISGINASVVNIQKEIDRLNEVAKNLNESLIDLQELGKYE | | | |
| | QYIKWP | | | |
| 13 | YECDIPIGAGICASYQTQTNSPRRAASVASQSIIAYTMSLGAENS | Protease cleavage | 127 | 660 to |
| | VAYSNNSIAIPTNFTISVTTEILPVSMTKTSVDCTMYICGDSTECS | domain S1/S2 with | | 786 |
| | NLLLQYGSFCTQLNRALTGIAVEQDKNTQEVFAQVK | R in the wildtype | | |
| | | peptide motif ARS | | |
| | | replaced by A | | |
| 14 | LPDPSKPSKASFIEDLL | S2′proteolytic site | 17 | 806 to |
| | | epitope with R in | | 822 |
| | | thewildtype | | |
| | | peptide motif KRS | | |
| | | replaced by A | | |
| 15 | QTNSPRRAASVASQSIIA | S1/S2 proteolytic | 18 | 677 to |
| | | site epitope with R | | 694 |
| | | in the wildtype | | |
| | | peptide motif ARS | | |
| | | replaced by A | | |
| 17 | MLLVNQSHQGFNKEHTSKMVSAIVLYVLLAAAAHSAFAADCV | SARS CoV-2 Wuhan | 1272 | |
| | NLTTRTQLPPAYTNSFTRGVYYPDKVFRSSVLHSTQDLFLPFFSN | (AA 15-1213 of WT) | | |
| | VTWFHAIHVSGTNGTKRFDNPVLPFNDGVYFASTEKSNIIRGW | RRAR to AGAG | | |
| | IFGTTLDSKTQSLLIVNNATNVVIKVCEFQFCNDPFLGVYYHKNN | DKVE replaced by | | |
| | KSWMESEFRVYSSANNCTFEYVSQPFLMDLEGKQGNFKNLREFV | DPPE | | |
| | FKNIDGYFKIYSKHTPINLVRDLPQGFSALEPLVDLPIGINITRF | C terminal His Tag | | |
| | QTLLALHRSYLTPGDSSSGWTAGAAAYYVGYLQPRTFLLKYNEN | 1-40 | | |
| | GTITDAVDCALDPLSETKCTLKSFTVEKGIYQTSNFRVQPTESIVR | gp67 signal | | |
| | FPNITNLCPFGEVFNATRFASVYAWNRKRISNCVADYSVLYNSA | | | |
| | SFSTFKCYGVSPTKLNDLCFTNVYADSFVIRGDEVRQIAPGQTG | | | |
| | KIADYNYKLPDDFTGCVIAWNSNNLDSKVGGNYNYLYRLFRKS | | | |
| | NLKPFERDISTEIYQAGSTPCNGVEGFNCYFPLQSYGFQPTNGV | | | |
| | GYQPYRVVVLSFELLHAPATVCGPKKSTNLVKNKCVNFNFNGL | | | |
| | TGTGVLTESNKKFLPFQQFGRDIADTTDAVRDPQTLEILDITPCS | | | |
| | FGGVSVITPGTNTSNQVAVLYQDVNCTEVPVAIHADQLTPTW | | | |
| | RVYSTGSNVFQTRAGCLIGAEHVNNSYECDIPIGAGICASYQTQ | | | |
| | TNSPAGAGSVASQSIIAYTMSLGAENSVAYSNNSIAIPTNFTISV | | | |
| | TTEILPVSMTKTSVDCTMYICGDSTECSNLLLQYGSFCTQLNRAL | | | |
| | TGIAVEQDKNTQEVFAQVKQIYKTPPIKDFGGFNFSQILPDPSK | | | |
| | PSKRSFIEDLLFNKVTLADAGFIKQYGDCLGDIAARDLICAQKFN | | | |
| | GLTVLPPLLTDEMIAQYTSALLAGTITSGWTFGAGAALQIPFAM | | | |
| | QMAYRFNGIGVTQNVLYENQKLIANQFNSAIGKIQDSLSSTASA | | | |
| | LGKLQDVVNQNAQALNTLVKQLSSNFGAISSVLNDILSRLDPPE | | | |
| | AEVQIDRLITGRLQSLQTYVTQQLIRAAEIRASANLAATKMSECV | | | |
| | LGQSKRVDFCGKGYHLMSFPQSAPHGVVFLHVTYVPAQEKNF | | | |
| | TTAPAICHDGKAHFPREGVFVSNGTHWFVTQRNFYEPQIITTD | | | |
| | NTFVSGNCDVVIGIVNNTVYDPLQPELDSFKEELDKYFKNHTSP | | | |
| | DVDLGDISGINASVVNIQKEIDRLNEVAKNLNESLIDLQELGKYE | | | |
| | QYIKWPGYIPEAPRDGQAYVRKDGEWVLLSTFLHHHHHH | | | |
| 19 | MLLVNQSHQGFNKEHTSKMVSAIVLYVLLAAAAHSAFAADCV | SARS CoV-2 Wuhan | 717 | |
| | NLTTRTQLPPAYTNSFTRGVYYPDKVFRSSVLHSTQDLFLPFFSN | (AA 15-685 of WT) | | |
| | VTWFHAIHVSGTNGTKRFDNPVLPFNDGVYFASTEKSNIIRGW | RRAR to AGAG | | |
| | IFGTTLDSKTQSLLIVNNATNVVIKVCEFQFCNDPFLGVYYHKNN | DKVE replaced by | | |
| | KSWMESEFRVYSSANNCTFEYVSQPFLMDLEGKQGNFKNLREFV | DPPE | | |
| | FKNIDGYFKIYSKHTPINLVRDLPQGFSALEPLVDLPIGINITRF | C terminal His Tag | | |
| | QTLLALHRSYLTPGDSSSGWTAGAAAYYVGYLQPRTFLLKYNEN | 1-40 | | |
| | GTITDAVDCALDPLSETKCTLKSFTVEKGIYQTSNFRVQPTESIVR | gp67 signal | | |
| | FPNITNLCPFGEVFNATRFASVYAWNRKRISNCVADYSVLYNSA | | | |
| | SFSTFKCYGVSPTKLNDLCFTNVYADSFVIRGDEVRQIAPGQTG | | | |
| | KIADYNYKLPDDFTGCVIAWNSNNLDSKVGGNYNYLYRLFRKS | | | |
| | NLKPFERDISTEIYQAGSTPCNGVEGFNCYFPLQSYGFQPTNGV | | | |
| | GYQPYRVVVLSFELLHAPATVCGPKKSTNLVKNKCVNFNFNGL | | | |
| | TGTGVLTESNKKFLPFQQFGRDIADTTDAVRDPQTLEILDITPCS | | | |
| | FGGVSVITPGTNTSNQVAVLYQDVNCTEVPVAIHADQLTPTW | | | |
| | RVYSTGSNVFQTRAGCLIGAEHVNNSYECDIPIGAGICASYQTQ | | | |
| | TNSPAGAGHHHHHH | | | |
| 21 | MLLVNQSHQGFNKEHTSKMVSAIVLYVLLAAAAHSAFAADRV | SARS CoV-2 Wuhan | 269 | |
| | QPTESIVRFPNITNLCPFGEVFNATRFASVYAWNRKRISNCVAD | (AA 319-541 of WT) | | |
| | YSVLYNSASFSTFKCYGVSPTKLNDLCFTNVYADSFVIRGDEVRQ | C terminal His Tag | | |
| | IAPGQTGKIADYNYKLPDDFTGCVIAWNSNNLDSKVGGNYNYL | 1-40 | | |
| | YRLFRKSNLKPFERDISTEIYQAGSTPCNGVEGFNCYFPLQSYGF | gp67 signal | | |
| | QPTNGVGYQPYRVVVLSFELLHAPATVCGPKKSTNLVKNKCVN | | | |
| | FHHHHHH | | | |
| 23 | MLLVNQSHQGFNKEHTSKMVSAIVLYVLLAAAAHSAFAADRV | SARS CoV-2 Wuhan | 319 | |
| | QPTESIVRFPNITNLCPFGEVFNATRFASVYAWNRKRISNCVAD | (AA 319-591 of WT) | | |
| | YSVLYNSASFSTFKCYGVSPTKLNDLCFTNVYADSFVIRGDEVRQ | C terminal His Tag | | |
| | IAPGQTGKIADYNYKLPDDFTGCVIAWNSNNLDSKVGGNYNYL | 1-40 | | |
| | YRLFRKSNLKPFERDISTEIYQAGSTPCNGVEGFNCYFPLQSYGF | gp67 signal | | |
| | QPTNGVGYQPYRVVVLSFELLHAPATVCGPKKSTNLVKNKCVN | | | |
| | FNFNGLTGTGVLTESNKKFLPFQQFGRDIADTTDAVRDPQTLEI | | | |
| | LDITPCSHHHHHH | | | |
| 25 | MRFPSIFTAVLFAASSALAAPVNTTTEDETAQIPAEAVIGYSDLE | SARS CoV-2 Wuhan | 285 | |
| | GDFDVAVLPFSNSTNNGLLFINTTIASIAAKEEGVSLEKRNITNLC | (AA 331-524 of WT) | | |
| | PFGEVFNATRFASVYAWNRKRISNCVADYSVLYNSASFSTFKCY | C terminal His Tag | | |
| | GVSPTKLNDLCFTNVYADSFVIRGDEVRQIAPGQTGKIADYNYK | 1-85 alpha- | | |
| | LPDDFTGCVIAWNSNNLDSKVGGNYNYLYRLFRKSNLKPFERDI | secretion signal | | |
| | STEIYQAGSTPCNGVEGFNCYFPLQSYGFQPTNGVGYQPYRVV | | | |
| | VLSFELLHAPATVHHHHHH | | | |
| 26 | MFVFLVLLPLVSSQCVNLTTRTQLPPAYTNSFTRGVYYPDKVFR | Full length SARS- | 1273 | |
| | SSVLHSTQDLFLPFFSNVTWFHAIHVSGTNGTKRFDNPVLPFN | CoV-2 Spike | | |
| | DGVYFASTEKSNIIRGWIFGTTLDSKTQSLLIVNNATNWIKVCE | protein, DKVE | | |
| | FQFCNDPFLGVYYHKNNKSWMESEFRVYSSANNCTFEYVSQP | replaced by DPPE | | |
| | FLMDLEGKQGNFKNLREFVFKNIDGYFKIYSKHTPINLVRDLPQ | 1-12 secretion | | |
| | GFSALEPLVDLPIGINITRFQTLLALHRSYLTPGDSSSGWTAGAA | signal | | |
| | AYYVGYLQPRTFLLKYNENGTITDAVDCALDPLSETKCTLKSFTV | | | |
| | EKGIYQTSNFRVQPTESIVRFPNITNLCPFGEVFNATRFASVYA | | | |
| | WNRKRISNCVADYSVLYNSASFSTFKCYGVSPTKLNDLCFTNVY | | | |
| | ADSFVIRGDEVRQIAPGQTGKIADYNYKLPDDFTGCVIAWNSN | | | |
| | NLDSKVGGNYNYLYRLFRKSNLKPFERDISTEIYQAGSTPCNGVE | | | |
| | GFNCYFPLQSYGFQPTNGVGYQPYRVVVLSFELLHAPATVCGP | | | |
| | KKSTNLVKNKCVNFNFNGLTGTGVLTESNKKFLPFQQFGRDIA | | | |
| | DTTDAVRDPQTLEILDITPCSFGGVSVITPGTNTSNQVAVLYQD | | | |
| | VNCTEVPVAIHADQLTPTWRVYSTGSNVFQTRAGCLIGAEHVN | | | |
| | NSYECDIPIGAGICASYQTQTNSPRRARSVASQSIIAYTMSLGAE | | | |
| | NSVAYSNNSIAIPTNFTISVTTEILPVSMTKTSVDCTMYICGDSTE | | | |
| | CSNLLLQYGSFCTQLNRALTGIAVEQDKNTQEVFAQVKQIYKTP | | | |
| | PIKDFGGFNFSQILPDPSKPSKRSFIEDLLFNKVTLADAGFIKQYG | | | |
| | DCLGDIAARDLICAQKFNGLTVLPPLLTDEMIAQYTSALLAGTIT | | | |
| | SGWTFGAGAALQIPFAMQMAYRFNGIGVTQNVLYENQKLIA | | | |
| | NQFNSAIGKIQDSLSSTASALGKLQDVVNQNAQALNTLVKQLS | | | |
| | SNFGAISSVLNDILSRL [symbol] AEVQIDRLITGRLQSLQTYVTQQL | | | |
| | RAAEIRASANLAATKMSECVLGQSKRVDFCGKGYHLMSFPQSA | | | |
| | PHGVVFLHVTYVPAQEKNFTTAPAICHDGKAHFPREGVFVSNG | | | |
| | THWFVTQRNFYEPQIITTDNTFVSGNCDVVIGIVNNTVYDPLO | | | |
| | PELDSFKEELDKYFKNHTSPDVDLGDISGINASVVNIQKEIDRLN | | | |
| | EVAKNLNESLIDLQELGKYEQYIKWPWYIWLGFIAGLIAIVMVTI | | | |
| | MLCCMTSCCSCLKGCCSCGSCCKFDEDDSEPVLKGVKLHYT | | | |
| 27 | VNLTTRTQLPPAYTNSFTRGVYYPDKVFRSSVLHSTQDLFLPFFS | Full length S1/S2 | 1198 | |
| | NVTWFHAIHVSGTNGTKRFDNPVLPFNDGVYFASTEKSNIIRG | spike ectodomain, | | |
| | WIFGTTLDSKTQSLLIVNNATNVVIKVCEFQFCNDPFLGVYYHK | DKVE replaced by | | |
| | NNKSWMESEFRVYSSANNCTFEYVSQPFLMDLEGKQGNFKNLRE | DPPE | | |
| | FVFKNIDGYFKIYSKHTPINLVRDLPQGFSALEPLVDLPIGINIT | | | |
| | RFQTLLALHRSYLTPGDSSSGWTAGAAAYYVGYLQPRTFLLKYN | | | |
| | ENGTITDAVDCALDPLSETKCTLKSFTVEKGIYQTSNFRVQPTESI | | | |
| | VRFPNITNLCPFGEVFNATRFASVYAWNRKRISNCVADYSVLYN | | | |
| | SASFSTFKCYGVSPTKLNDLCFTNVYADSFVIRGDEVRQIAPGQT | | | |
| | GKIADYNYKLPDDFTGCVIAWNSNNLDSKVGGNYNYLYRLFRK | | | |
| | SNLKPFERDISTEIYQAGSTPCNGVEGFNCYFPLQSYGFQPTNG | | | |
| | VGYQPYRVVVLSFELLHAPATVCGPKKSTNLVKNKCVNFNFNG | | | |
| | LTGTGVLTESNKKFLPFQQFGRDIADTTDAVRDPQTLEILDITPC | | | |
| | SFGGVSVITPGTNTSNQVAVLYQDVNCTEVPVAIHADQLTPTW | | | |
| | RVYSTGSNVFQTRAGCLIGAEHVNNSYECDIPIGAGICASYQTQ | | | |
| | TNSPRRARSVASQSIIAYTMSLGAENSVAYSNNSIAIPTNFTISVT | | | |
| | TEILPVSMTKTSVDCTMYICGDSTECSNLLLQYGSFCTQLNRALT | | | |
| | GIAVEQDKNTQEVFAQVKQIYKTPPIKDFGGFNFSQILPDPSKP | | | |
| | SKRSFIEDLLFNKVTLADAGFIKQYGDCLGDIAARDLICAQKFNG | | | |
| | LTVLPPLLTDEMIAQYTSALLAGTITSGWTFGAGAALQIPFAMQ | | | |
| | MAYRFNGIGVTQNVLYENQKLIANQFNSAIGKIQDSLSSTASAL | | | |
| | GKLQDVVNQNAQALNTLVKQLSSNFGAISSVLNDILSRL [symbol] A | | | |
| | EVQIDRLITGRLQSLQTYVTQQLIRAAEIRASANLAATKMSECVL | | | |
| | GQSKRVDFCGKGYHLMSFPQSAPHGVVFLHVTYVPAQEKNFT | | | |
| | TAPAICHDGKAHFPREGVFVSNGTHWFVTQRNFYEPQIITTDN | | | |
| | TFVSGNCDVVIGIVNNTVYDPLQPELDSFKEELDKYFKNHTSPD | | | |
| | VDLGDISGINASVVNIQKEIDRLNEVAKNLNESLIDLQELGKYEQ | | | |
| | YIKWP | | | |
| 28 | MFVFLVLLPLVSSQCVNLTTRTQLPPAYTNSFTRGVYYPDKVFR | Full length SARS- | 1273 | |
| | SSVLHSTQDLFLPFFSNVTWFHAIHVSGTNGTKRFDNPVLPFN | CoV-2 Spike protein | | |
| | DGVYFASTEKSNIIRGWIFGTTLDSKTQSLLIVNNATNVVIKVCE | with R in the | | |
| | FQFCNDPFLGVYYHKNNKSWMESEFRVYSSANNCTFEYVSQP | wildtype peptide | | |
| | FLMDLEGKQGNFKNLREFVFKNIDGYFKIYSKHTPINLVRDLPQ | motif ARS or KRS | | |
| | GFSALEPLVDLPIGINITRFQTLLALHRSYLTPGDSSSGWTAGAA | replaced by A, DKVE | | |
| | AYYVGYLQPRTFLLKYNENGTITDAVDCALDPLSETKCTLKSFTV | replaced by DPPE | | |
| | EKGIYQTSNFRVQPTESIVRFPNITNLCPFGEVFNATRFASVYA | 1-12secretion | | |
| | WNRKRISNCVADYSVLYNSASFSTFKCYGVSPTKLNDLCFTNVY | signal | | |
| | ADSFVIRGDEVRQIAPGQTGKIADYNYKLPDDFTGCVIAWNSN | | | |
| | NLDSKVGGNYNYLYRLFRKSNLKPFERDISTEIYQAGSTPCNGVE | | | |
| | GFNCYFPLQSYGFQPTNGVGYQPYRVVVLSFELLHAPATVCGP | | | |
| | KKSTNLVKNKCVNFNFNGLTGTGVLTESNKKFLPFQQFGRDIA | | | |
| | DTTDAVRDPQTLEILDITPCSFGGVSVITPGTNTSNQVAVLYQD | | | |
| | VNCTEVPVAIHADQLTPTWRVYSTGSNVFQTRAGCLIGAEHVN | | | |
| | NSYECDIPIGAGICASYQTQTNSPRRAASVASQSIIAYTMSLGAE | | | |
| | NSVAYSNNSIAIPTNFTISVTTEILPVSMTKTSVDCTMYICGDSTE | | | |
| | CSNLLLQYGSFCTQLNRALTGIAVEQDKNTQEVFAQVKQIYKTP | | | |
| | PIKDFGGFNFSQILPDPSKPSKASFIEDLLFNKVTLADAGFIKQYG | | | |
| | DCLGDIAARDLICAQKFNGLTVLPPLLTDEMIAQYTSALLAGTIT | | | |
| | SGWTFGAGAALQIPFAMQMAYRFNGIGVTQNVLYENQKLIA | | | |
| | NQFNSAIGKIQDSLSSTASALGKLQDVVNQNAQALNTLVKQLS | | | |
| | SNFGAISSVLNDILSRL [symbol] AEVQIDRLITGRLQSLQTYVTQQLI | | | |
| | RAAEIRASANLAATKMSECVLGQSKRVDFCGKGYHLMSFPQSA | | | |
| | PHGVVFLHVTYVPAQEKNFTTAPAICHDGKAHFPREGVFVSNG | | | |
| | THWFVTQRNFYEPQIITTDNTFVSGNCDVVIGIVNNTVYDPLQ | | | |
| | PELDSFKEELDKYFKNHTSPDVDLGDISGINASVVNIQKEIDRLN | | | |
| | EVAKNLNESLIDLQELGKYEQYIKWPWYIWLGFIAGLIAIVMVTI | | | |
| | MLCCMTSCCSCLKGCCSCGSCCKFDEDDSEPVLKGVKLHYT | | | |
| 29 | VNLTTRTQLPPAYTNSFTRGVYYPDKVFRSSVLHSTQDLFLPFFS | Full length S1/S2 | 1198 | |
| | NVTWFHAIHVSGTNGTKRFDNPVLPFNDGVYFASTEKSNIIRG | spike ectodomain | | |
| | WIFGTTLDSKTQSLLIVNNATNVVIKVCEFQFCNDPFLGVYYHK | with R in the | | |
| | NNKSWMESEFRVYSSANNCTFEYVSQPFLMDLEGKQGNFKNLRE | wildtype peptide | | |
| | FVFKNIDGYFKIYSKHTPINLVRDLPQGFSALEPLVDLPIGINIT | motif ARS or KRS | | |
| | RFQTLLALHRSYLTPGDSSSGWTAGAAAYYVGYLQPRTFLLKYN | replaced by A, DKVE | | |
| | ENGTITDAVDCALDPLSETKCTLKSFTVEKGIYQTSNFRVQPTESI | replaced by DPPE | | |
| | VRFPNITNLCPFGEVFNATRFASVYAWNRKRISNCVADYSVLYN | | | |
| | SASFSTFKCYGVSPTKLNDLCFTNVYADSFVIRGDEVRQIAPGQT | | | |
| | GKIADYNYKLPDDFTGCVIAWNSNNLDSKVGGNYNYLYRLFRK | | | |
| | SNLKPFERDISTEIYQAGSTPCNGVEGFNCYFPLQSYGFQPTNG | | | |
| | VGYQPYRVVVLSFELLHAPATVCGPKKSTNLVKNKCVNFNFNG | | | |
| | LTGTGVLTESNKKFLPFQQFGRDIADTTDAVRDPQTLEILDITPC | | | |
| | SFGGVSVITPGTNTSNQVAVLYQDVNCTEVPVAIHADQLTPTW | | | |
| | RVYSTGSNVFQTRAGCLIGAEHVNNSYECDIPIGAGICASYQTQ | | | |
| | TNSPRRAASVASQSIIAYTMSLGAENSVAYSNNSIAIPTNFTISVT | | | |
| | TEILPVSMTKTSVDCTMYICGDSTECSNLLLQYGSFCTQLNRALT | | | |
| | GIAVEQDKNTQEVFAQVKQIYKTPPIKDFGGFNFSQILPDPSKP | | | |
| | SKASFIEDLLFNKVTLADAGFIKQYGDCLGDIAARDLICAQKFNG | | | |
| | LTVLPPLLTDEMIAQYTSALLAGTITSGWTFGAGAALQIPFAMQ | | | |
| | MAYRFNGIGVTQNVLYENQKLIANQFNSAIGKIQDSLSSTASAL | | | |
| | GKLQDVVNQNAQALNTLVKQLSSNFGAISSVLNDILSRLDPPEA | | | |
| | EVQIDRLITGRLQSLQTYVTQQLIRAAEIRASANLAATKMSECVL | | | |
| | GQSKRVDFCGKGYHLMSFPQSAPHGVVFLHVTYVPAQEKNFT | | | |
| | TAPAICHDGKAHFPREGVFVSNGTHWFVTQRNFYEPQIITTDN | | | |
| | TFVSGNCDVVIGIVNNTVYDPLQPELDSFKEELDKYFKNHTSPD | | | |
| | VDLGDISGINASVVNIQKEIDRLNEVAKNLNESLIDLQELGKYEQ | | | |
| | YIKWP | | | |
| 30 | QAGSTPCNGVEGFNCYFPLQSYGFQPTNGVGYOPGSGSQAGS | ConCat 1 | 344 | |
| | TPCNGVEGFNCYFPLQSYGFQPTNGVGYQPGSGSQAGSTPCN | | | |
| | GVEGFNCYFPLQSYGFQPTNGVGYQPGSGSQAGSTPCNGVEG | | | |
| | FNCYFPLQSYGFQPTNGVGYQPGSGSQAGSTPCNGVEGFNCY | | | |
| | FPLQSYGFQPTNGVGYQPGSGSQAGSTPCNGVEGFNCYFPLQ | | | |
| | SYGFQPTNGVGYQPGSGSQAGSTPCNGVEGFNCYFPLQSYGF | | | |
| | QPTNGVGYQPGSGSQAGSTPCNGVEGFNCYFPLQSYGFQPTN | | | |
| | GVGYQPGSGSQAGSTPCNGVEGFNCYFPLQSYGFQPTNGVGY | | | |
| | QPHHHHHH | | | |
| 31 | QAGSTPGNGVEGSGSQAGSTPGNGVEGSGSQAGSTPGNGVE | ConCat 2 | 302 | |
| | GSGSQAGSTPGNGVEGSGSQAGSTPGNGVEGSGSQAGSTPG | | | |
| | NGVEGSGSQAGSTPGNGVEGSGSQAGSTPGNGVEGSGSQAG | | | |
| | STPGNGVEGSGSQAGSTPGNGVEGSGSQAGSTPGNGVEGSG | | | |
| | SQAGSTPGNGVEGSGSQAGSTPGNGVEGSGSQAGSTPGNGV | | | |
| | EGSGSQAGSTPGNGVEGSGSQAGSTPGNGVEGSGSQAGSTP | | | |
| | GNGVEGSGSQAGSTPGNGVEGSGSQAGSTPGNGVEGSGSQA | | | |
| | GSTPGNGVEHHHHHH | | | |
| 32 | QPTNGVGYQPGSGSQPTNGVGYQPGSGSQPTNGVGYQPGS | ConCat 3 | 324 | |
| | GSQPTNGVGYQPGSGSQPTNGVGYQPGSGSQPTNGVGYQP | | | |
| | GSGSQPTNGVGYQPGSGSQPTNGVGYQPGSGSQPTNGVGY | | | |
| | QPGSGSQPTNGVGYQPGSGSQPTNGVGYQPGSGSQPTNGV | | | |
| | GYQPGSGSQPTNGVGYQPGSGSQPTNGVGYQPGSGSQPTNG | | | |
| | VGYQPGSGSQPTNGVGYQPGSGSQPTNGVGYQPGSGSQPTN | | | |
| | GVGYQPGSGSQPTNGVGYQPGSGSQPTNGVGYQPGSGSQPT | | | |
| | NGVGYQPGSGSQPTNGVGYQPGSGSQPTNGVGYQPHHHHH | | | |
| | H | | | |
| 33 | GFNGYFPLQSYGSGSGFNGYFPLQSYGSGSGFNGYFPLQSYGS | ConCat 4 | 333 | |
| | GSGFNGYFPLQSYGSGSGFNGYFPLQSYGSGSGFNGYFPLQSY | | | |
| | GSGSGFNGYFPLQSYGSGSGFNGYFPLQSYGSGSGFNGYFPLQ | | | |
| | SYGSGSGFNGYFPLQSYGSGSGFNGYFPLQSYGSGSGFNGYFPL | | | |
| | QSYGSGSGFNGYFPLQSYGSGSGFNGYFPLQSYGSGSGFNGYF | | | |
| | PLQSYGSGSGFNGYFPLQSYGSGSGFNGYFPLQSYGSGSGFNG | | | |
| | YFPLQSYGSGSGFNGYFPLQSYGSGSGFNGYFPLQSYGSGSGF | | | |
| | NGYFPLQSYGSGSGFNGYFPLQSYGHHHHHH | | | |
| 34 | SNNLDSKVGGNYGSGSSNNLDSKVGGNYGSGSSNNLDSKVGG | ConCat 5 | 322 | |
| | NYGSGSSNNLDSKVGGNYGSGSSNNLDSKVGGNYGSGSSNNL | | | |
| | DSKVGGNYGSGSSNNLDSKVGGNYGSGSSNNLDSKVGGNYGS | | | |
| | GSSNNLDSKVGGNYGSGSSNNLDSKVGGNYGSGSSNNLDSKV | | | |
| | GGNYGSGSSNNLDSKVGGNYGSGSSNNLDSKVGGNYGSGSSN | | | |
| | NLDSKVGGNYGSGSSNNLDSKVGGNYGSGSSNNLDSKVGGNY | | | |
| | GSGSSNNLDSKVGGNYGSGSSNNLDSKVGGNYGSGSSNNLDS | | | |
| | KVGGNYGSGSSNNLDSKVGGNYHHHHHH | | | |
| 35 | QTNSPRRARSVASQSIGSGSQTNSPRRARSVASQSIGSGSQTNS | ConCat 6 | 302 | |
| | PRRARSVASQSIGSGSQTNSPRRARSVASQSIGSGSQTNSPRRA | | | |
| | RSVASQSIGSGSQTNSPRRARSVASQSIGSGSQTNSPRRARSVA | | | |
| | SQSIGSGSQTNSPRRARSVASQSIGSGSQTNSPRRARSVASQSI | | | |
| | GSGSQTNSPRRARSVASQSIGSGSQTNSPRRARSVASQSIGSGS | | | |
| | QTNSPRRARSVASQSIGSGSQTNSPRRARSVASQSIGSGSQTNS | | | |
| | PRRARSVASQSIGSGSQTNSPRRARSVASQSIHHHHHH | | | |
| 36 | QAGSTPCNGVEGFNCYFPLQSYGFQPTNGVGYOP | Building block for | 34 | |
| | | ConCat 1 | | |
| 37 | QAGSTPGNGVE | Building block for | 11 | |
| | | ConCat 2, PCN | | |
| | | replaced by PGN | | |
| 38 | QPTNGVGYQP | Building block for | 10 | |
| | | ConCat 3 | | |
| 39 | GFNGYFPLQSYG | Building block for | 12 | |
| | | ConCat 4, NCY | | |
| | | replaced by NGY | | |
| 40 | SNNLDSKVGGNY | Building block for | 12 | |
| | | ConCat 5 | | |
| 41 | QTNSPRRARSVASQSI | Building block for | 16 | |
| | | ConCat 6 | | |

TABLE 1

| SEQ ID | | length | | | | | | | | | | | | | |
|---|---|---|---|---|---|---|---|---|---|---|---|---|---|---|---|
| NO | Type | (AA residues) | prefered max length of protein or peptide comprising the SEQ ID NO | | | | | | | | | | | | |
| 1 | Full length SARS-CoV-2 | 1273 | 6400 | 5100 | 3800 | 2550 | 2400 | 2300 | 2150 | 2050 | 1900 | 1800 | 1650 | 1500 | 1400 |
| | Spike protein | | | | | | | | | | | | | | |
| 2 | Full length S1/S2 spike | 1198 | 6000 | 4800 | 3600 | 2400 | 2300 | 2150 | 2050 | 1900 | 1800 | 1700 | 1550 | 1450 | 1300 |
| | ectodomain | | | | | | | | | | | | | | |
| 3 | Binding Domain | 274 | 1400 | 1100 | 850 | 550 | 520 | 490 | 470 | 440 | 410 | 380 | 360 | 330 | 300 |
| 4 | Shorter fragment with | 202 | 1010 | 810 | 600 | 400 | 380 | 360 | 340 | 320 | 300 | 280 | 260 | 240 | 220 |
| | binding domain | | | | | | | | | | | | | | |
| 5 | Protease cleavage | 127 | 360 | 508 | 381 | 254 | 241 | 229 | 216 | 203 | 191 | 178 | 165 | 152 | 140 |
| | domain S1/S2 | | | | | | | | | | | | | | |
| 6 | S2′proteolytic site | 17 | 85 | 68 | 51 | 34 | 32 | 31 | 29 | 27 | 26 | 24 | 22 | 20 | 19 |
| | epitope | | | | | | | | | | | | | | |
| 7 | S1/S2 proteolytic site | 18 | 90 | 72 | 54 | 36 | 34 | 32 | 31 | 29 | 27 | 25 | 23 | 22 | 20 |
| | epitope | | | | | | | | | | | | | | |
| 8 | Binding motif loop 1a | 11 | 55 | 44 | 33 | 22 | 21 | 20 | 19 | 18 | 17 | 15 | 14 | 13 | 12 |
| | epitope | | | | | | | | | | | | | | |
| 9 | Binding motif loop 1b | 10 | 50 | 40 | 30 | 20 | 19 | 18 | 17 | 16 | 15 | 14 | 13 | 12 | 11 |
| | epitope | | | | | | | | | | | | | | |
| 10 | Binding motif loop 2 | 11 | 55 | 44 | 33 | 22 | 21 | 20 | 19 | 18 | 17 | 15 | 14 | 13 | 12 |
| | epitope | | | | | | | | | | | | | | |

What is claimed is:

1. A method for isolating or producing antibodies, the method comprising:
immunizing a female vertebrate animal by administration of at least one protein or peptide comprising at least one amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41,
collecting one or more lactation or ovulation product from the animal after immunization, and
isolating or purifying at least one antibody from the products,
wherein the protein or peptide is, comprises, or is a fragment of, the SARS CoV 2 spike protein

2. The method according to claim 1, wherein the animal is a female bird.

3. The method according to claim 1 or 2, wherein the animal is an anseriform bird.

4. The method according to any one of the aforementioned claims, wherein the animal is a chicken.

5. The method according to any one of the aforementioned claims, wherein the ovulation product that is collected from the animal is one or more eggs.

6. The method according to any one of the aforementioned claims, wherein the antibody that is purified from the eggs is IgY.

7. The method according to any one of the aforementioned claims, wherein the animal is a female mammal.

8. The method according to any one of the aforementioned claims, wherein the animal is a ruminant.

9. The method according to any one of the aforementioned claims, wherein the animal is a cow, pig, camel, horse, donkey, goat or sheep.

10. The method according to any one of the aforementioned claims, wherein the lactation product collected from the animal is, whey, milk or colostrum.

11. The method according to any one of the aforementioned claims, wherein the antibody is at least one of IgA, IgG, and IgM.

12. The method according to any one of the aforementioned claims, wherein the protein or peptide comprising at least one amino acid sequence selected from the group consisting of any one of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41 has a maximum length as set forth in table 1.

13. The method according to any one of the aforementioned claims, wherein two or more proteins or peptides comprising at least one amino acid sequence selected from the group consisting of any one of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41 are used for immunizing the animal.

14. The method according to any one of the aforementioned claims, wherein
a) at least one single chain protein or peptide is used for immunizing which comprises two or more subsequences each comprising an amino acid sequence selected from the group consisting of any one of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, and/or
b) at least one homo- or heterodimer, -oligomer or -multimer is used for immunizing which comprises two or more chains each comprising an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO 29.

15. The method according to any one of the aforementioned claims, wherein at least one protein or peptide used for immunizing is conjugated to a second molecule known to be immunogenic in the animal being immunized.

16. The method according to any one of claims 1-6 and 12-15, wherein the isolation or purification of the antibody comprises the steps of
a) removal of the bulk of the lipids and lipoproteins from the yolk (“delipidation”), and
b) concentration or purification of the IgY fraction.

17. The method according to any one of claims 1-6 and 12-16, wherein prior to immunization, one or more eggs of the bird which is to be immunized are collected.

18. The method according to any one of claims 1-6 and 12-17, wherein immunization takes place by means of injection of the protein or peptide into the breast tissue of the bird.

19. The method according to any one of claims 1-6 and 12-18, wherein for immunization, a dosis of between ≥0.02 and ≤0.5 mg protein or peptide is injected to the bird.

20. The method according to any one of claims 1 and 6-19, wherein the isolation or purification of the antibody comprises the steps of
a) optionally, skimming milk or colostrum by removing fat
b) removing casein e.g. by lowering pH to 4.6 by adding an acid, and
c) collecting the liquid phase and concentration or purification of the antibody fraction.

21. The method according to any one of claims 1 and 6-20, wherein, prior to immunization, one or more samples of milk, colostrum or whey of the mammal which is to be immunized are collected.

22. The method according to any one of claims 1 and 6-21, wherein immunization takes place by means of injection of the protein or peptide by at least one of
intramuscular
subcutaneous
intramammary or
intravenous
infusion or injection.

23. The method according to any one of claims 1 and 6-22, wherein for immunization, a dosis of between ≥0.05 and ≤2 mg protein or peptide is injected to the mammal

24. The method according to any one of the aforementioned claims, wherein for immunization, an adjuvant is co-administered with the at least one protein or peptide.

25. The method according to any one of the aforementioned claims, wherein immunization is repeated between one and 10 times.

26. The method according to any one of the aforementioned claims, wherein, in the repeated immunization, the amount of the protein or peptide is reduced to between ≥70% and ≤10% of the initial dosis.

27. The method according to any one of the aforementioned claims, wherein immunization is performed by in at least two steps, wherein,
c) in a first step, the bird or mammal is immunized by administration of at least one single chain protein or peptide which comprises two or more subsequences each comprising an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, and
d) in a second step performed after the first step the bird or mammal is immunized by administration of at least one monomer which comprises an amino acid sequence selected from the group consisting of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO 29.

28. The method according to any one of the aforementioned claims, wherein at least one of the proteins or peptides used for immunization is produced by at least one of peptide synthesis or recombinant expression.

29. The method according to claim 28, wherein the recombinant expression is done in at least one of
a prokaryotic expression system, like e.g. E. coli or B. subtilis
a fungal or yeast based expression system, like S. cerevisiae
a protozoan expression system, like Tetrahymena thermophila
an baculovirus/insect expression system
a mammalian expression system, like CHO cells.

30. An antibody that binds specifically to a protein or peptide comprising a sequence selected from the group consisting of any one of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, wherein the protein or peptide is, comprises or is a fragment of the SARS CoV-2 spike protein.

31. A combination of two or more antibodies that bind specifically to one or more proteins or peptides selected from the group consisting of any one of any one of SEQ ID NO: 1-SEQ ID NO: 15, SEQ ID NO: 17, SEQ ID NO: 19, SEQ ID NO: 21, SEQ ID NO: 23, SEQ ID NO: 25 or any one of SEQ ID NO: 26-SEQ ID NO: 41, wherein the proteins or peptides are, comprise or are a fragment of the SARS CoV-2 spike protein.

32. The antibody or antibodies according to any one of claim 30 or 31, which
inhibits binding of SARS-Cov2 to the human ACE2 receptor, and/or
interferes with the priming of the spike protein by blocking proteolytic cleavage thereof at S1/S2 or S2.

33. The antibody or antibodies according to any one of claims 30-32, which has/have a neutralizing titer (NT₅₀) of ≤1×10⁵.

34. The antibody or antibodies according to any one of claims 30-33, which competes with ACE2 for binding to the SARS-Cov2 spike protein.

35. The antibody or antibodies according to any one of claims 30-34, which is/are polyclonal antibody/antibodies.

36. The antibody or antibodies according to any one of claims 30-35, which is/are produced by the method according to any one of claims 1-29.

37. A product comprising at least one antibody according to any one of claims 30-36, the product being provided as at least one selected from the group consisting of
Lozenge
Chewing gum
Tablet
Cream or gel
Gargling solution or mouthwash
liquid solution for topic application
aerosol for intraoral, intratracheal or intranasal administration, and/or
aqueous solution for intravenous, subcutaneous or intramuscular administration.

38. A product comprising at least one antibody according to any one of claims 30-36, the product being embedded or provided in at least one item selected from the group consisting of:
Breathing mask
Filter for fluid media, including gases and liquids, and/or
Disinfectant gel, liquid, aerosol or spray.

39. The antibody or antibodies according to any one of claims 30-36 for use in the treatment of a patient that suffers from or diagnosed for SARS Cov-2 infection, or for the prevention of such condition.

40. The product according to any one of claims 37-38 for use in the treatment of a patient that suffers from or diagnosed for SARS Cov-2 infection, or for the prevention of such condition.

41. The antibody or antibodies or product according to any one of the aforementioned claims, the antibody or antibodies being co-administered with, or the product further comprising, at least one of
a protease inhibitor, preferably an inhibitor of TMPRSS2 or an inhibitor of the CoV-2 C30 endopeptidase (also called Mpro or 3CLpro)
a nucleoside or nucleotide analogue, preferably which is accepted by viral RNA-dependent RNA polymerase
an IL-6 antagonist,
a soluble angiotensin-converting enzyme 2, and/or
a peptide that comprises a sequence that is homologous to the S1/S2 sequence of the SarsCov2 spike protein,
wherein co-administration can be carried out concomitantly or consecutively.
